(19) United States
(12) Patent Application Publication
(10) Pub. No.: US 2023/0372284 A1
(43) Pub. Date: Nov. 23, 2023

(54) METHODS FOR TREATING ATRIAL FIBRILLATIONS

(71) Applicant: Xyra, LLC, Santa Rosa, CA (US)

(72) Inventors: Pascal Druzgala, Santa Rosa, CA (US); Peter Gerard Milner, Mountain View, CA (US)

(21) Appl. No.: 18/221,235

(22) Filed: Jul. 12, 2023

Related U.S. Application Data
(63) Continuation-in-part of application No. 17/978,835, filed on Nov. 1, 2022, now Pat. No. 11,759,640.
(60) Provisional application No. 63/525,093, filed on Jul. 5, 2023.
(60) Provisional application No. 63/334,852, filed on Apr. 26, 2022.
(60) Provisional application No. 63/297,426, filed on Jan. 7, 2022.
(60) Provisional application No. 63/437,543, filed on Jan. 6, 2023.

(51) Int. Cl. A61K 31/343; A61B 5/024

(52) U.S. Cl. CPC A61K 31/343; A61B 5/02438

(57) ABSTRACT

Patients with either paroxysmal or persistent AFib and whose untreated AFib is greater than a threshold level of AFib are qualified as eligible for treatment with a drug, (e.g., budiodarone) capable of controlling heart rhythm in the patient. Qualified patients are treated with the drug while their heart rates are monitored with a wearable, and if a given dose of the drug is deemed ineffective, the dose is adjusted until an effective dose is determined for the patient of the patient is deemed non-responsive to the drug and removed from therapy.

### CROSS REFERENCE TO RELATED APPLICATIONS

This application claims benefit of U.S. Provisional 63/437,543 filed on Jan. 6, 2023 and U.S. Provisional 63/525,093 filed on Jul. 5, 2023, the contents of which are herein incorporated in their entirety. This application is a continuation-in-part of U.S. Non-provisional patent application Ser. No. 17/978,835, filed Nov. 1, 2022, which claims the benefit of U.S. Provisional Application No. 63/334,852, filed Apr. 26, 2022, and U.S. Provisional Application No. 63/297,426, filed Jan. 7, 2022, the contents of which are herein incorporated in their entirety.

### FIELD

Disclosed are methods for treating atrial fibrillation (AFib) in patients diagnosed with paroxysmal or persistent atrial fibrillation (AFib) who are determined to potentially benefit from pharmacotherapy.

### STATE OF THE ART

AFib is a serious medical condition characterized by an abnormal heart rhythm often at accelerated heart rates. There are three basic types of AFib, paroxysmal, persistent, and permanent. Paroxysmal AFib and persistent AFib are characterized by spontaneous periods of AFib that occur between periods of sinus rhythm, whereas permanent AFib is characterized by a constant state of atrial fibrillation that can only be treated indirectly with medications such as blood thinners.

Untreated paroxysmal and persistent AFib lead to an increased risk of strokes, congestive heart failure, and death. These increased risks vary with the extent of AFib in a given patient. Variability in the increased risk is due to the extent of AFib in a given patient. Untreated patients suffering from either paroxysmal or persistent AFib will see a progression of their disease over time from paroxysmal to persistent or from persistent to permanent AFib respectively.

Some patients with paroxysmal or persistent AFib have short fleeting episodes of AFib each of which are less than 1 hour in duration and/or have a cumulative AFib burden over any 24-hour period of less than 5 hours. Such patients have what will be referred to herein as de minimis AFib. These patients have a sufficiently lower risk of strokes, congestive heart failure, and death than similar patients having longer or more frequent episodes of AFib. Patients with permanent AFib bear the highest risk of stroke, congestive heart failure, and death and typically do not respond to medical intervention.

Current pharmacotherapy for patients with AFib does not aim to control their AFib but, rather, minimizes the associated risk of stroke due to clot formation associated with AFib by, for example, prescribing blood thinners. The use of such blood thinners is not a panacea but comes with a price. The underlying rationale for prescribing the use of blood thinners is that they carry less risk of harm to patients with AFib than does untreated AFib itself even though the risk of harm using blood thinners is significant. According to the report cited by Market Insider, QuarterWatch's 2016 Annual Report Issue (Institute for Safe Medication Practices. (2016). Quarterwatch: Monitoring FDA MedWatch reports. Annual Report Issue), anticoagulant drug use accounted for the most adverse events of all prescription or over-the-counter drugs in 2016, specifically 21,996 severe injuries in the U.S., including 3,018 reported deaths. In clinical trials, anticoagulants have repeatedly demonstrated high injury rates, causing bleeding in 8% to 19% of patients treated for a year. The Quarterwatch 2016 report estimates that 6.3% of patients exposed to an anticoagulant for one year will require an emergency department visit due to the drug's adverse effects, with 3.1% requiring hospitalization.

About two-thirds of all AFib patients are symptomatic with symptoms ranging from mild to debilitating. Physical symptoms include but are not limited to, general fatigue, fatigue when exercising or during exertion, faintness, light-headedness, confusion, a rapid and/or irregular heartbeat, fluttering or thumping in the chest, shortness of breath, dizziness, weakness, confusion, sweating, chest pain and/or pressure. However, regardless of the extent of any physical symptoms, the corresponding mental anxiety concerning potential cardiac issues can be severe and can be manifested by psychological symptoms such as anxiety, stress, and/or depression in the patient. In turn, these psychological symptoms aggravate the AFib in the patient and, in their own right, are serious health issues. As such, the ability to reduce the symptoms of AFib is an important aspect of treating symptomatic patients with AFib.

Regardless of whether the patient's AFib is symptomatic or asymptomatic, studies have shown that the risk of stroke and/or heart failure is independent of whether the patient suffers symptoms associated with AFib. See, e.g., Flaker, et al., “Asymptomatic Atrial Fibrillation: Demographic Features and Prognostic Information from the Atrial Fibrillation Follow-Up Investigation of Rhythmic Management (AFFIRM) Study,” American Heart Journal, April 2005, pages 657-663, which is incorporated herein by reference in its entirety. A patient's recognition or lack thereof of periodic AFib episodes does not correlate to a differential risk of stroke or congestive heart failure. Rather, it suggests that symptomatic patients are more aware of their condition and are more likely to seek medical intervention.

The difficulty with the conventional medical intervention of paroxysmal and persistent AFib is that the sporadic and unpredictable nature of each episode of AFib does not allow the clinician to properly evaluate the extent of the patient's AFib or the efficacy of the drugs used to treat it. Unlike conventional pharmacotherapy where a drug's efficacy is measured by using standardized tests that measure specific targets after a period of time from the start of therapy (e.g., cholesterol tests), a clinician cannot use the electrocardiogram (EKG) information gathered during a conventional office visit to determine how many episodes of AFib, the duration for each AFib episode, or the AFib burden that the patient has endured since the prior office visit.

Accordingly, there remain several problems in the treatment of patients with paroxysmal and persistent AFib using pharmacotherapy. These include the inability of the clinician to assess: the extent of a patient's AFib, which is needed to determine whether the patient's AFib warrants treatment with the pharmacotherapy; whether a given drug is efficacious for a given patient; at what dosage the drug is efficacious for the patient; and whether a patient on the pharmacotherapy is or becomes non-responsive to the drug and should be removed from the pharmacotherapy.

### SUMMARY

Disclosed are methods for identifying and qualifying a patient for pharmacotherapy with a drug for controlling heart rhythm wherein the identified and qualified patients are most likely to benefit from such a drug. In the methods described herein, the very low risk of stroke or heart failure in patients with de minimis AFib does not warrant treatment, because the risks associated with pharmacotherapy may be greater in such patients. Likewise, patients with permanent AFib are deemed unlikely to benefit from said pharmacotherapy. The methods, therefore, identify patients with either paroxysmal or persistent AFib as eligible for further evaluation to determine whether their AFib exceeds a de minimis level (discussed in more detail below). At the completion of the evaluation, patients with either permanent AFib or de minimis AFib have been disqualified from pharmacotherapy, thereby providing a carefully defined subset of pharmacotherapy-qualified patients.

Patients with AFib are evaluated with heart rhythm monitoring over an extended time period of at least 7 days or a period sufficient to provide a baseline level of AFib (e.g., a baseline level of symptoms associated with AFib, a baseline number of AFib episodes over the period and/or a baseline AFib burden during the period) in the absence of any pharmacotherapy. Typically, this evaluation period (referred to herein as a qualification period) during which the patient's monitoring is conducted is at least 1 week, at least 2 weeks, but longer periods of 1 month (e.g., 4 weeks) or longer can be considered. Longer periods may give a more accurate measure of the patient's level of AFib. Patients determined to have either de minimis or permanent AFib are disqualified and the remaining patients are deemed eligible or qualified for pharmacotherapy.

In some embodiments, the methods comprise: a) a qualification protocol based on the heart rhythm data generated by a wearable over a qualification period to assess if a patient is likely to benefit from pharmacotherapy; b) a treatment protocol to determine and maintain an effective dosage of the drug for the patient. The treatment protocol employs a wearable that assesses the heart rhythm data of the patient to assess efficacy of one or more doses of the drug administered in an iterative process until an efficacious dose is achieved or, if not, to remove the patient from treatment; and c) using wearable to monitor the heart rhythm data of qualified patients being administered an efficacious dose of the drug to ensure that the dosage administered remains efficacious or, if not, dose adjusting the amount of drug administered to either a new efficacious dosage is achieved or, if not, determining that the patient is now refractory to said therapy and removing the patient from said pharmacotherapy.

In one embodiment, qualified patients start pharmacotherapy at a first (approved) dosage of the drug. The first dosage of the drug may be selected by the attending clinician predicated on the patient's baseline data. In one embodiment, the first dosage is a lowest approved dosage. In another embodiment, the first dosage is any approved dosage less than a maximum approved dosage. Heart rhythms, and optionally symptoms associated with AFib, are monitored for patients on pharmacotherapy. Monitoring of heart rhythm is conducted with a wearable (e.g., as described herein) to assess the efficacy of the pharmacotherapy.

Once monitoring is initiated, the patient's heart rhythm (and optionally symptoms associated with AFib) are evaluated to assess whether the drug is efficacious at that dosage. The minimum evaluation period time duration and the minimum temporal resolution should be selected to accurately characterize the patient's AFib level at that dosage. The evaluation period time duration should be sufficient to obtain a stable concentration of the drug in vivo. For example, the evaluation period time duration may be at least 3 days, at least 1 week, and at least 2 weeks after the initiation of pharmacotherapy. Also, or alternatively, the minimum evaluation time period may begin when the drug has achieved a steady state blood level (e.g., at least 2-3 days after initiation of budiodarone pharmacotherapy) and have a length of at least 1 day, at least 3 days, at least 1 week, and at least 2 weeks, etc. Also, or alternatively, the evaluation time period may begin once the level of the patient's AFib has reached a steady state (e.g., does not vary substantially between successive evaluation periods). Qualified patients who evidence an efficacious result at a given dosage are maintained on that dosage. Also, or alternatively, qualified patients who evidence an efficacious result to the pharmacotherapy at a given dosage may be dose adjusted one or more times by decreasing the dosage to identify the lowest efficacious dosage. Qualified patients who do not evidence an efficacious result at the first dosage (or at a given dosage) are dose adjusted one or more times by increasing the dosage of the drug until either the patient evidences an efficacious result (e.g., by reducing AFib episode durations, maximum durations, number of episodes over a given duration, AFib burden, etc., and/or by reducing a risk of stroke and/or congestive heart failure, as disclosed elsewhere herein), or the patient fails to evidence an efficacious result even at the highest approved dosage. Such a patient is deemed non-responsive to the drug and is therefore disqualified and excluded from the pharmacotherapy.

In some cases, patients who have achieved an efficacious result at a given dosage may experience side effects, and/or may be at risk for side effects, from the drug. The clinician may decide based on such side effects and/or risks to dose adjust by decreasing the dosage with the goal of retaining efficacy while reducing side effects or the risk of side effects.

These methods provide for a process to identify a unique and exclusive population of patients who would be most likely to benefit from pharmacotherapy with a drug for controlling heart rhythm (e.g., budiodarone). Such a population of patients would not be identified by conventional methods. The methods define a subset of patients by excluding patients who have permanent AFib, which eliminates about 50% of patients with AFib. Additionally, these methods define a more restrictive subset of patients by further excluding patients identified as having de minimis AFib (as described herein). Patients with de minimis AFib have a limited risk of stroke and/or heart failure, making pharmacotherapy aimed to reduce such risks by adjusting heart rhythm of limited benefit. The restrictive subset of patients is then qualified for pharmacotherapy with the drug.

In another embodiment, qualified patients on pharmacotherapy with the drug are evaluated for drug efficacy at increasing dosages of the drug until a therapeutic outcome is achieved or until the patient is deemed to be a non-responder and is excluded from the therapy. Central to this approach is continued monitoring of the patient's heart rhythm (and optionally symptoms associated with AFib) to assess if and when the patient evidences a therapeutic response to the drug. The monitoring is performed over a time scale and at a temporal resolution sufficient to accurately assess a level of AFib in the qualified patient during treatment. Those patients who achieve an efficacious result from a dosage of the drug represent a unique patient subpopulation who are responsive to the pharmacotherapy. Patient monitoring is continued for the responsive patient during therapy to ensure that a given dosage of the drug, determined to be efficacious for the patient, remains efficacious. If at any point a dosage of the drug is determined to no longer be efficacious for the patient, then a dose adjustment for the patient will occur. Dose adjusting with continued monitoring is performed for a pharmacotherapy-qualified patient if or until a patient becomes non-responsive to the therapy and is removed from therapy.

The methods described herein provide a new paradigm in the treatment of AFib in a subclass of patients that addresses the underlying sporadic and unpredictable nature of the disease. Patients who benefit from pharmacotherapy are identified, while those who would not benefit from the therapy are excluded. Moreover, patients who initially show drug efficacy but later become refractory to that drug can be identified and later excluded from the therapy. So, unlike current therapy for AFib, for which the efficacy of any drug is difficult or impossible to measure, the methods defined herein continually establish efficacy in a specifically defined AFib patient subpopulation by excluding patients pre-therapy who are unlikely to benefit from the pharmacotherapy with monitoring and then excluding non-responsive patients during the pharmacotherapy with monitoring.

A significant advantage achieved by the methods described herein is that using these methods as a first course of treatment for qualified patients could allow for referring only patients who are excluded from the pharmacotherapy (e.g., pre-therapy or after initiating the pharmacotherapy) for electrophysiological intervention (ablation) to treat their AFib, if their AFib is sufficiently serious to warrant ablation. The targeted treatment of patients most likely to benefit from the pharmacotherapy and dynamic definition of the qualified patient population even after initiation of therapy avoids unnecessary use of the exceptionally expensive and invasive ablation procedure on patients who are responsive to the significantly less expensive and less invasive pharmacotherapy.

In one embodiment, the pharmacotherapy comprises the administration of budiodarone, which has the IUPAC nomenclature of ([(2S)-butan-2-yl] 2-[3-[4-[2-(diethylamino)ethoxy]-3,5-diiodobenzoyl]-1-benzofuran-2-yl]acetate), or a pharmaceutically acceptable salt thereof. Budiodarone is a compound demonstrated to be useful in reducing stroke risk in patients with paroxysmal and/or persistent AFib, including patients with AFib refractory to other treatments—see, e.g., U.S. Pat. No. 9,549,912, which is incorporated herein by reference in its entirety.

In one embodiment, a patient's qualification period described herein provides the clinician with a baseline evaluation of the extent of both long episodes of AFib (longer than about 5 or about 5.5 hours) and/or an AFib burden of over 5 hours in any given 24-hour period. For a patient administered budiodarone at a given dosage, an efficacious result is determined if the patient evidences one or more of the following relative to corresponding baseline evaluation data over a comparison period beginning with the initiation of budiodarone administration at said dosage:
a) a reduction of the number of AFib episodes longer than about 5 hours or about 5.5 hours wherein said reduction reduces such AFib episodes by at least 30% over the comparison period; or
b) a reduction in the maximum AFib burden in any 24-hour segment has been reduced by at least 30% over the comparison period; or
c) a reduction in symptoms over the comparison period, wherein the patient is a symptomatic patient which symptoms are associated with AFib.

In one embodiment, symptomatic qualified patients with AFib are at risk of anxiety, stress, and/or depression due to their diagnosis and/or symptoms. An efficacious result can also be a reduction in the aggregated level of symptoms experienced by the patient. A reduction in symptoms and/or aggregate symptoms in the patient can be evaluated using the procedures of Example 9, for example.

The comparison period recited above is designed to provide a temporal limit for determining the efficacy of the pharmacotherapy which, in the absence of such a limit, could be construed to be indefinitely long extending for years or decades. Hence, for the purposes herein, a comparison period is a duration of time, after the start of efficacy monitoring as described above, over which heart rhythm data acquired from the patient (e.g., via a wearable fitted to and/or employed on the patient) is analyzed to compare with previous heart rhythm data and/or AFib data of the patient (e.g., baseline data acquired during the qualification period, and/or data acquired during previous a comparison period data). In an embodiment, the comparison period is at least as long as the qualification period described herein, or based on a different period of time such as daily, weekly, and at least about 2-weeks (e.g., 13, 14, or 15 days) or twice, three or four times as long (i.e., at least about 4 weeks, at least about 6 weeks, or at least about 8 weeks). In an example, the comparison period can be a length that at least extends beyond a time at which the drug level has substantially stabilized in the patient (e.g., at least 2-3 days, 7 days, 14 days, etc. in a patient provided budiodarone), or can have a shorter length (e.g., continuously, or 1 day or more) if the comparison period begins after drug levels have substantially stabilized in the patient, (e.g., beginning 2 days or beginning 3 days after initiation of administering budiodarone at a given dosage).

Recognizing that AFib is a progressive disease even in cases where efficacious pharmacotherapy is employed, comparison periods that extend beyond 6 months are not necessarily probative of the efficacy of the drug as one cannot distinguish between disease progression and drug efficacy (akin to cancer chemotherapeutics). Accordingly, comparison periods in some embodiments are daily, weekly, 2 weeks, 4 weeks, 6 weeks, 8 weeks, 12 weeks, and up to but not exceeding 24 weeks. Once a first comparison period is completed, the efficacy of the pharmaceutical can be established. Subsequent comparison periods are then used a) to ensure that the dose of the pharmaceutical remains efficacious for responsive patients; orb) to establish efficacy at a higher dose for patients not responsive to the current dose employed. If a patient is not responsive to the pharmaceutical used at the highest approved dose in the comparison period, the patient is then removed from treatment with the pharmaceutical. In such cases, the patient may be referred to electrophysiology for treatment (e.g., ablation).

There is medical data indicating that a meaningful reduction in the risk of stroke is achieved for patients having no episodes of AFib that extend beyond 5.5 hours. See, e.g., Capucci (Capucci, A., et al. (2005). “Monitored atrial fibrillation duration predicts arterial embolic events in patients suffering from bradycardia and atrial fibrillation implanted with anti-tachycardia pacemakers.” Journal of the American College of Cardiology, 46(10), 1913-1920) and the TRENDS study (Glotzer, et al, (2009). The relationship between daily atrial tachyarrhythmia burden from implantable device diagnostics and stroke risk: the TRENDS study. Circulation: Arrhythmia and Electrophysiology. 2(5), 474-480), the contents of which are incorporated herein in their entirety. In an embodiment, budiodarone therapy is based on an approved dose that reduces such long episodes during a comparison period by at least 50%, and, more preferably, at least 80% and, even more preferably, that eliminates such long episodes during a comparison period. In another embodiment, budiodarone therapy is based on an approved dose that reduces long episodes of AFib that extend beyond 1 hour by at least 50% and, preferably, at least 80% during a comparison period and, more preferably, eliminates such long episodes during a comparison period. In any case, the ability to limit episodes of AFib during a comparison period that extends beyond 5.5 hours or 1 hour reduces the patient's risk of stroke, and the elimination of such episodes during a comparison period moves the patient into a perceived “safer harbor” or perhaps better stated as a “safer harbor”.

Accordingly, in one embodiment, there is provided a method of treating a patient diagnosed with either paroxysmal or persistent atrial fibrillation (AFib) and qualified for treatment with budiodarone, said method comprising:
a) administering a first dose of budiodarone to said qualified patient while employing a wearable to monitor to collect heart rhythm data of said qualified patient, wherein said first dose is an approved therapeutic dose that is less than a maximum approved dose of budiodarone;
b) monitoring said qualified patient for efficacy of the administered first dose of budiodarone to assess whether said first dose is efficacious for said patient during a comparison period of 1 or more days, provided that assessing the efficacy of budiodarone is delayed for at least 7 or 14 days after start of therapy;
c) if the first dose is assessed as not efficacious for said patient by the end of the comparison period, dose adjusting the amount of budiodarone one or more times as necessary to achieve an efficacious result using at least the same delay and comparison period as in b), provided that the adjusted dose does not exceed the maximum approved dose; or, if an efficacious result is not achieved when evaluated at the maximum approved dose, removing said patient from budiodarone therapy; and
d) if an administered dose of the first dose or an adjusted dose is efficacious for the patient, continuing to monitor the patient's heart rhythm to ensure that the administered dose of budiodarone remains efficacious, provided that if said administered dose is no longer efficacious step c) is repeated with said administered dose as the first dose.

In an embodiment, said patient has been qualified for treatment with budiodarone based on at least one AFib episode of at least about 5 hours or at least about 5.5 hours over at least 7 or 14 days during which said patient is not administered budiodarone therapy. Also, or alternatively said patient has been qualified for treatment with budiodarone based on having a baseline level of AFib burden of at least 5 hours over at least 7 or 14 days during which said patient is not administered budiodarone therapy.

In one embodiment, the monitoring is conducted in a continuous manner during the administration of the budiodarone at each evaluated dosage while assessing the presence of long duration AFib episodes such that the patient is notified that such is or has occurred.

In one embodiment, the comparison period can be daily, weekly, biweekly, over 1 month, over 2 months, over 3 months, over 4 months, over 5 months, or up to 6 months while monitoring is done continuously. In another embodiment, to better characterize a patient as having permanent AFib, the evaluation period is extended to 4, 6, 8, 12 weeks, or longer.

In one embodiment, episodes of AFib over at least about 5 hours or 5.5 hours are tracked (e.g., counted, recorded, used in determining the efficacy of an administered drug, used in alerting a patient and/or clinician if a given number of such episodes occur or if such episodes occur with a given frequency indicating the patient is at risk for stroke and/or congenital heart failure). In another embodiment, episodes of AFib over at least about 5.5 hours are tracked.

In one embodiment, symptoms associated with AFib are evaluated by the patient and reported (e.g., using a scorecard or its equivalent) to indicate the number and/or severity of the patient's symptoms on a daily basis or as they occur.

In one embodiment, if an AFib episode of at least about 5 hours or at least about 5.5 hours or more is detected during any of the qualification periods, the comparison period, or during any later monitoring of the patient to assess the efficacy of a given dose, an alert or alarm is output to the patient (e.g., via wearable monitoring the heart rhythm data of the patient being analyzed to assess AFib episodes, or a computing device communicatively connected to the wearable), a clinician assessing the patient's AFib (e.g., via a computing device accessible to the clinician and/or receiving heart rhythm data from a wearable on the patient) and/or a central analysis center receiving data from the wearable of the patient.

The use of an alert is optionally available and can be provided immediately after the detection of a long duration AFib episode to provide the clinician and/or the patient with knowledge of the event. The comparison period is independent of the alert and provides data that allows the clinician to compare the efficacy of the drug over time against baseline. In one embodiment, the comparison period is differentiated from immediate notification (alarm) to the patient and/or the attending clinician of a long duration AFib episode that may require immediate attention. While the comparison period is intended to evaluate efficacy of the pharmaceutical (e.g., budiodarone) as compared to baseline, immediate notification is to alert the patient that possible medical attention should be sought.

In one embodiment, there is provided a method of treating a patient diagnosed with either paroxysmal or persistent atrial fibrillation (AFib) and qualified for treatment with budiodarone, said method comprising:
for a patient who has been qualified for treatment with budiodarone, wherein said qualification requires that said patient experiences at least one episode of long duration AFib of at least 1 hour during a qualification period or has an AFib burden of at least 2.5% during the entire qualification period, wherein said qualification period is at least 2 weeks during which the patient is not treated with said pharmaceutical;
a) administering a first dose of said pharmaceutical to a qualified patient while employing a wearable to monitor to collect heart rhythm data of said qualified patient, wherein said first dose is an approved therapeutic dose which is less than a maximum approved dose for said pharmaceutical;
b) monitoring said patient for efficacy of the administered first dose of said pharmaceutical to assess whether said first dose is efficacious for said patient during a comparison period of 1 or more days provided that assessing the efficacy of said pharmaceutical is delayed for at least 7 or 14 days after start of therapy;
c) if the administered first dose is assessed as not efficacious for said patient by the end of the comparison period, dose adjusting the amount of said pharmaceutical one or more times as necessary to achieve an efficacious result using at least the same delay and comparison period as in b), provided that the adjusted dose does not exceed the maximum approved dose; or if an efficacious result is not achieved when evaluated at the maximum approved dose, removing said patient from said pharmaceutical therapy; and
d) if an administered dose of the first dose or an adjusted dose is efficacious for the patient, continuing to monitor the patient's heart rhythm to ensure that the administered dose of said pharmaceutical remains efficacious, provided that if said administered dose is no longer efficacious, step c) is repeated with said administered dose as the first dose.

In one embodiment, there is provided a method of treating a patient diagnosed with either paroxysmal or persistent atrial fibrillation (AFib) and qualified for treatment pharmacotherapy using a pharmaceutical that inhibits episodes of AFib and/or reduces the duration of AFib episodes, said method comprising: for the patient who has been qualified for treatment with said pharmaceutical, wherein said qualification requires that said patient experiences at least one episode of long duration AFib of at least 1 hour during a qualification period or has an AFib burden of at least 2.5% during the entire qualification period, wherein said qualification period is at least 2 weeks during which the patient is not treated with said pharmaceutical;
a) administering a first dose of said pharmaceutical to a qualified patient while employing a wearable to monitor to collect heart rhythm data of said qualified patient, wherein said first dose is an approved therapeutic dose which is less than a maximum approved dose for said pharmaceutical;
b) monitoring said patient for efficacy of the administered first dose of said pharmaceutical to assess whether said first dose is efficacious for said patient during a comparison period of 1 or more days provided that assessing the efficacy of said pharmaceutical is delayed for at least 7 or 14 days after start of therapy;
c) if the administered first dose is assessed as not efficacious for said patient by the end of the comparison period, dose adjusting the amount of said pharmaceutical one or more times as necessary to achieve an efficacious result using at least the same delay and comparison period as in b), provided that the adjusted dose does not exceed the maximum approved dose; or if an efficacious result is not achieved when evaluated at the maximum approved dose, removing said patient from said pharmaceutical therapy; and
d) if an administered dose of the first dose or an adjusted dose is efficacious for the patient, continuing to monitor the patient's heart rhythm to ensure that the administered dose of said pharmaceutical remains efficacious, provided that if said administered dose is no longer efficacious, step c) is repeated with said administered dose as the first dose.

In one embodiment, there is provided a method of treating a patient diagnosed with either paroxysmal or persistent atrial fibrillation (AFib) and qualified for pharmacotherapy with a pharmaceutical, said method comprising:
a) administering a first dose of said pharmaceutical to said qualified patient while employing a wearable to monitor to collect heart rhythm data of said qualified patient, wherein said first dose is an approved therapeutic dose that is less than a maximum approved dose for said pharmaceutical;
b) monitoring said patient for efficacy of the administered first dose of said pharmaceutical to assess whether said dose is efficacious for said patient during a comparison period of 1 or more days, provided that assessing the efficacy of said pharmaceutical is delayed for at least 7 or 14 days after start of therapy with said pharmaceutical;
c) if the first dose is assessed as not efficacious for said patient by the end of the comparison period, dose adjusting the amount of said pharmaceutical one or more times as necessary to achieve an efficacious result using at least the same delay and comparison period as in c), provided that dose adjustment does not exceed the maximum approved dose; or, if an efficacious result is not achieved when evaluated at the maximum approved dose, removing said patient from said pharmacotherapy; and
d) if an administered dose of the first dose or an adjusted dose is efficacious for the patient, continuing to monitor the patient's heart rhythm to ensure that the administered dose of budiodarone remains efficacious, provided that if said administered dose is no longer efficacious step c) is repeated with said administered dose as the first dose.

In an embodiment, said patient has been qualified for pharmacotherapy with said pharmaceutical based on at least one AFib episode of at least about 5 hours or at least about 5.5 hours over at least 7 or 14 days during which said patient is not administered said pharmaceutical. In another embodiment, said patient has been qualified for pharmacotherapy with said pharmaceutical based on having a baseline level of AFib burden of at least 5 hours over at least 7 or 14 days during which said patient is not administered said pharmaceutical.

In one embodiment, there is provided a method of treating a patient diagnosed with either paroxysmal or persistent atrial fibrillation (AFib) and qualified for treatment with budiodarone which inhibits episodes of AFib and/or the duration of AFib episodes, said method comprising: for a patient who has been qualified for treatment with budiodarone wherein said qualification requires that said patient experiences at least one episode of long duration AFib of at least 1 hour or has an AFib burden of at least 2.5% during the entire qualification period wherein said qualification period is at least 2 weeks during which the patient is not treated with budiodarone;
a) administering a first dose of said pharmaceutical to a qualified patient while employing a wearable to monitor to collect heart rhythm data of said qualified patient, wherein said first dose is an approved therapeutic dose that is less than the maximum approved dose;
b) monitoring said patient with the wearable for efficacy of budiodarone administered at said first therapeutic dose to assess whether said dose is efficacious for each of said patients during an evaluation period;
c) for patients who did not achieve an efficacious result at the prior dose of budiodarone at the end of the evaluation period, dose adjusting the amount of budiodarone one or more times as necessary to achieve an efficacious result using at least the length of the evaluation period to assess efficacy provided that dose adjustment does not exceed the maximum approved dose; or if an efficacious result is not achieved when evaluated at the maximum approved dose, removing said patient from budiodarone therapy; and
d) for patients who did achieve an efficacious results, continuing to monitor their heart rhythm to ensure that the dose of said pharmaceutical remains efficacious provided that if said dose is no longer efficacious repeat the protocol of c) as needed.

In one embodiment for the methods described above, the qualification period and the evaluation period are at least about 7 days, at least about 14 days, or at least about 28 days.

In one embodiment, for the methods described herein, the at least one episode of long duration AFib is at least about 5.5 hours, or at least about 5 hours, or at least about 1 hour, or at least about 30 minutes.

In one embodiment, for the methods described herein, the at least one episode of long duration AFib is at least about 5 hours during a 24-hour period.

In one embodiment, for the methods described herein, the monitoring of said patient on budiodarone is continuous during said therapy.

In one embodiment, the monitoring of said patient on said pharmaceutical is periodically conducted provided that during said periodic monitoring with the periodic monitoring being conducted throughout said period.

The methods described herein can be used to reduce the risk of strokes in patients with paroxysmal or persistent AFib wherein these methods employ a pharmaceutical, including budiodarone, that inhibits episodes of AFib and/or the duration of AFib episodes, in the manner set forth herein. Patients at risk of strokes with paroxysmal or persistent AFib especially include those at elevated risk of strokes such as those who previously had a stroke, diabetic patients, patients with high blood pressure, patients over 75 years of age or over 80 years of age, and the like.

The methods described herein can be used to reduce the risk of congestive heart failure in patients with paroxysmal or persistent AFib wherein these methods employ a pharmaceutical, including budiodarone, that inhibits episodes of AFib and/or the duration of AFib episodes, in the manner set forth herein. Patients with paroxysmal or persistent AFib and at risk of congestive heart failure especially include those at elevated risk of congestive heart failure such as diabetic patients, patients with high blood pressure, patients with valvular heart disease, and the like.

Patients with elevated risk for strokes and/or congestive heart failure can be treated according to the methods described herein even if said patients are categorized as having de minimis AFib and/or if they are not qualified before treatment, or if they are qualified over a lower baseline level of AFib than would be used to qualify lower risk patients. For example, a patient with a relatively low risk for stroke and/or congenital heart failure (e.g., may be qualified for budiodarone therapy

In an embodiment, a method of treating a patient diagnosed with either paroxysmal or persistent atrial fibrillation (AFib) and at an elevated risk for strokes and/or congestive heart failure may include:
(a) monitoring heart rhythm data of the patient, wherein the heart rhythm data may be collected over an extended period of time using a wearable,
(b) administering budiodarone (or another pharmaceutical) to said patient at a first dose, wherein said first dose is an approved therapeutic dose which is less than the maximum approved dose for budiodarone (or the other pharmaceutical); and
(c) monitoring said patient for efficacy of the first administered dose of budiodarone (or the other pharmaceutical) to assess whether said dose is efficacious for said patient during a comparison period of 1 or more days provided that assessing the efficacy of budiodarone (or the other pharmaceutical) is delayed for at least 7 or 14 days after start of therapy. Said first dose may be the lowest approved dose. A dose may be assessed as efficacious for said patient if, over the comparison period, it eliminates or reduces the number of episodes of AFib of at least about an hour, or at least about 0.5 hours, for example, or otherwise substantially reducing a risk for stroke or congestive heart failure in the patient. For patients who did not achieve an efficacious result at the first dose of budiodarone (or the other pharmaceutical) by the end of the comparison period, the method further comprises a step (d) of dose adjusting the amount of budiodarone (or the other pharmaceutical) one or more times as necessary to achieve an efficacious result using the same delay and comparison period, or longer, to assess efficacy, provided that dose adjustment does not exceed the maximum approved dose. An efficacious result can be determined as described herein. If, at any point, an efficacious result is not achieved when evaluated at the maximum approved dose, the method may further comprise a step (e) removing said patient from budiodarone (or the other pharmaceutical) therapy. For patients who did achieve an efficacious result, the method may further comprise a step (f) continuing to monitor their heart rhythm to ensure that the dose of budiodarone (or the other pharmaceutical) remains efficacious. If said dose is no longer efficacious in said patient, the steps of (d) dose adjusting or, if no efficacious result is achieved, (e) removing the patient from budiodarone (or the other pharmaceutical) therapy are repeated. This method may be performed on a patient with any level of paroxysmal or persistent AFib, provided said patient is at elevated risk for stroke and/or congestive heart failure. In an embodiment, the patient is qualified for budiodarone therapy, as described herein.

In an embodiment, a patient with paroxysmal or persistent AFib and at an elevated risk of stroke and/or congestive heart can be treated by pharmacotherapy according to the methods described herein. Budiodarone, or another pharmaceutical capable of reducing numbers and/or durations of AFib episodes, may be administered in combination with a blood thinner (e.g., an anticoagulant). For example, an attending clinician may further administer a blood thinner in combination with budiodarone to mitigate the risk of stroke or cardiac heart failure in a patient.

In an embodiment, a method of treating a patient diagnosed with either paroxysmal or persistent atrial fibrillation (AFib) and at an elevated risk for strokes and/or congestive heart failure may include: (a) monitoring heart rhythm data of the patient, wherein the heart rhythm data may be collected over an extended period of time using a wearable, (b) administering both a blood thinner and budiodarone to said patient, wherein the budiodarone is administered at a first dose, wherein said first dose is an approved therapeutic dose which is less than the maximum approved dose for budiodarone; and (c) monitoring said patient for efficacy of the first administered dose of budiodarone to assess whether said dose is efficacious for said patient during a comparison period of 1 or more days provided that assessing the efficacy of budiodarone is delayed for at least 7 or 14 days after start of therapy. Said first dose of budiodarone may be a lowest approved dose. For patients who did not achieve an efficacious result at the first dose of budiodarone administered by the end of the comparison period, the method further comprises (d) dose adjusting the amount of budiodarone one or more times as necessary to achieve an efficacious result using the same delay and comparison period, or longer, to assess efficacy, provided that dose adjustment does not exceed the maximum approved dose. An efficacious result may be determined as described herein. If, at any point, an efficacious result is not achieved when evaluated at the maximum approved dose, the method further comprises (e) removing said patient from budiodarone therapy. For patients who did achieve an efficacious result, the method further comprises (f) continuing to monitor their heart rhythm to ensure that the dose of budiodarone remains efficacious. If said dose is no longer efficacious, the steps of (d) dose adjusting or, if no efficacious result is achieved, (e) removing the patient from therapy are repeated. This method may be performed on a patient with any level of paroxysmal or persistent AFib, provided said patient is at elevated risk for stroke and/or congestive heart failure. In an embodiment, the patient is qualified for budiodarone therapy, as described herein.

### BRIEF DESCRIPTION OF THE DRAWINGS

FIG. 1 illustrates a chart indicating how the patient's AFib data is used in conjunction with treatment with budiodarone; and

FIG. 2 illustrates a cardiogram of a patient with intermittent AFib.

### DETAILED DESCRIPTION

This disclosure is directed to methods for monitoring the heart rhythm of patients diagnosed with atrial fibrillation (AFib) with a wearable. Such monitoring allows for therapeutic intervention coupled with the ability to modulate the dosing of the pharmaceutical employed to obtain therapeutic end points. The following terms are defined below. Terms that are not defined are given their definition in context or are given their medically acceptable definition.

The terminology used herein is for the purpose of describing particular embodiments only and is not intended to be limiting. As used herein, the singular forms “a”, “an”, and “the” are intended to include the plural forms as well, unless the context clearly indicates otherwise.

### Definitions

As used herein, the term “optional” or “optionally” means that the subsequently described event or circumstance may or may not occur and that the description includes instances where the event or circumstance occurs and instances where it does not.

As used herein, the term “about” when used before a numerical designation, e.g., temperature, time, amount, concentration, and such other, including a range, indicates approximations that may vary by (+) or (−) 15%, 10%, 5%, 1%, or any subrange and/or value there between. Preferably, the term “about” when used with regard to a dose amount means that the dose may vary by +/−10%.

As used herein, the term “comprising” or “comprises” is intended to mean that the compositions or methods include the recited elements, but do not exclude others.

As used herein, the term “consisting essentially of” when used to define compositions and methods, shall mean excluding other elements of any essential significance to the stated purpose of the combination. Thus, a composition consisting essentially of the elements as defined herein, or a method consisting essentially of the protocol defined herein, would not exclude other components that do not materially affect the basic and novel characteristic(s) of the claimed subject matter.

As used herein, the term “consisting of” shall mean excluding more than trace elements of other ingredients or substantial method steps. Embodiments defined by each of these transition terms are within the scope of this disclosure.

As used herein, the term “AFib” or “atrial fibrillation” refers to all variants of atrial fibrillation except permanent AFib. Such variants include but are not limited to paroxysmal AFib, persistent AFib, as well as paroxysmal AFib and persistent AFib with low CHA₂DS₂-VASc scores (2 or less) and high CHA₂DS₂-VASc scores (3 or higher). CHA₂DS₂-VASc scores stand for congestive heart failure (C—1 pt), hypertension (H—1 pt), age ≥75 (A—2 pts), diabetes (D—1 pt), stroke (S—2 pt), vascular disease (V—1 pt), age 65 to 74 (A—1 pt), and sex category (Sc—1 pt for female) and are clinical prediction rules for estimating the risk of stroke in people with non-rheumatic atrial fibrillation (AF). A CHA₂DS₂-VASc score comprises 1 point or 2 points for each of the constituent risk factors, as indicated above.

As used herein, the term “long duration AFib” relates to the length of time an episode of AFib lasts in a patient. This length relates to the risk of stroke and/or heart failure. As is apparent, the longer the length of time a patient is in AFib, the higher the risk becomes. So, in one embodiment, a long duration AFib is any AFib episode lasting over at least about 1 hour, provided that the AFib is not permanent. In turn, long duration AFib is further categorized by the subsets “medium-long duration” AFib, which is at least about 24 hours or less in duration (i.e., over at least about 1 hour and up to 24 hours) and “longer-duration” AFib, which includes AFib episodes of over 24 hours, but which are not permanent AFib.

As used herein, the term “de minimis AFib” refers to AFib in a patient with paroxysmal or persistent AFib where each episode lasts less than about 5.5 hours, less than about 5 hours, or less than about 1 hour in duration, and/or where the maximum AFib burden in any 24-hour period is less than about 5 hours. In a preferred embodiment, patients with de minimis AFib have sporadic episodes of AFib that each lasts less than about 1 hour, and/or the maximum AFib burden in any 24-hour monitoring period is less than about 3 hours. In another embodiment, the maximum AFib burden is less than about 2.5% of the total hours during any two-week period of patient monitoring.

As used herein, the term “paroxysmal AFib” refers to sporadic and intermittent episodes of AFib that last no more than 7 days before resolving back into sinus rhythm. Typically, but not necessarily, patients with de minimis AFib are also characterized as having paroxysmal AFib.

As used herein, the term “persistent AFib” refers to sporadic and intermittent episodes of AFib that last more than 7 days and may not resolve back into sinus rhythm without medical intervention (e.g., cardioversion).

As used herein, the term “permanent AFib” refers to consistent and prolonged duration of AFib with no apparent return to sinus rhythm, even with medical intervention (e.g., cardioversion). Permanent AFib is typically managed, not treated.

As used herein, the term “moderate AFib” refers to paroxysmal and persistent AFib that is not characterized as de minimis AFib.

As used herein, the term “approved dose” refers to a dose of the drug (i.e., budiodarone) that a regulatory body such as the US Food & Drug Administration has determined is suitable for use commercially or in clinical studies.

As used herein, the term “continuous” or “continuously” refers to monitoring conducted constantly during the engagement of a wearable by the patient. Included within the term “continuous” or “continuously” are wearables that constantly monitor the heart rhythm when worn and powered, but which may be taken off for limited periods of time (e.g., changing batteries, charging the wearable monitoring device, bathing, etc.).

As used herein, the term “semi-continuous” or “semi-continuously” refers to monitoring that is automatically done periodically by the wearable on a set schedule (e.g., every 15 seconds, every 30 seconds, every minute, and the like) without the patient's activation of the wearable. The set schedule may be a schedule designed to be able to monitor heart rhythm with a temporal resolution sufficient to detect AFib episodes and/or to allow the determination of lengths of long duration AFib episodes.

As used herein, the term “engageable” means that the monitor can be continuously on and/or attached to the patient, or the patient can place the wearable on and off his/her body and initiate monitoring without the need for assistance from an attending health care professional. As such, engageable wearables include devices on the skin and/or implanted subdermally into the patient.

As used herein, the term “monitoring the heart rhythm” means any evaluation that can be made of a patient's heart rhythm including the pulse rate, any aspect of the electric field of the patient's heart, and the like provided that such monitoring is capable of ascertaining when the patient is experiencing AFib.

As used herein, the term “initiate monitoring” includes both automatic initiation and physical initiation of patient monitoring. “Automatic initiation” occurs when the wearable, once placed on the patient, automatically starts monitoring without any further action by the patient. “Physical initiation” means that the patient is required to activate the monitoring by physically or verbally interacting with the wearable (e.g., pushing a button, providing a voice command, and the like).

As used herein, the term “transmission device” means any device capable of transmitting data from the wearable. The transmission device may be included in the wearable or may be a separate device that is in communication with the wearable. The separate device can be a smartphone, a computer such as a pad, a laptop, a desktop, and the like.

As used herein, the term “clinician” refers to a healthcare professional qualified to ascertain whether a patient's heart rhythm data correlates to sinus rhythm, atrial fibrillation, or other types of arrhythmias.

As used herein, the term “attending clinician” refers to the healthcare professional who is treating the patient with AFib. Such an attending clinician is typically a doctor or a nurse practitioner.

The term “directly” as it relates to the transmission of the patient's heart rhythm data refers to a transmission that is received by the attending clinician for evaluation regardless of whether the transmission is deposited in a Cloud site or through a number of servers, etc.

The term “indirectly” as it relates to the transmission of the patient's heart rhythm data refers to transmission to a clinician who evaluates the data and provides either the data or the conclusions reached regarding the data to the attending clinician or a health care provider. In such a case, the clinician can be a healthcare professional employed by a central analysis center or by the attending clinician, etc. to evaluate the heart rhythm data and provide instructions to the attending clinician either on an ongoing basis or when a change in the patient's condition warrants contact. In one embodiment, the clinician is employed by a central analysis center, a facility that reviews the data generated by its instruments and renders a diagnosis and/or recommendation that is transmitted to the attending clinician. The central analysis center has expertise in the instrument, the data generated, and the ability to analyze that data.

As used herein, the term “baseline” refers to a patient who has AFib and is monitored to determine the extent of AFib burden in any 24-hour period and the number of episodes of medium-long- and longer-duration AFib over a set period of time prior to treatment with budiodarone. In such a case, measuring the baseline before pharmacotherapy over a set period of time provides for the current state of the disease. In most if not all cases, the baseline state of the disease is unknown to the attending clinician and is necessary information to determine if subsequent treatment with pharmacotherapy is efficacious.

As used herein, the term “efficacious”, “efficacious result”, or language similar thereto refers to the treatment of a qualified patient having moderate AFib (or in certain conditions, such as for patients having an elevated risk for stroke and/or congestive heart failure, but indicating as having de minimis AFib) wherein said patient evidences a reduction in any one or a combination of any of the following due to said treatment:
a reduction in the number of long duration (including longer-duration) episodes of AFib as compared to baseline;
a reduction in the maximum AFib burden in a 24-hour period as compared to baseline; and/or
for patients with symptomatic AFib, a reduction in the symptoms associated therewith.

The term “reduction” in combination with an efficacious result (e.g., as above) refers to a reduction of the number of long duration AFib episodes, the extent of AFib burden, and/or the extent of AFib symptoms relative to corresponding baseline data of the patient by a desired amount (e.g., at least 10%, preferably at least 20%, more preferably at least 30%, more preferably at least 40%, more preferably at least 50%, etc.). The desired amount of reduction for an efficacious result in a patient may be selected to reduce a risk of stroke and/or CHF to a desired level. For example, previously mentioned Capucci and the Trends study demonstrate that a risk of stroke in AFib patients with AFib episodes having durations between 5.5-24 hours is 2.2 times that of individuals without AFib, and a risk of stroke in AFib patients with AFib episodes having durations greater than 24 hours is 3.1 times that of individuals without AFib. Therefore, a desired amount of reduction may be to eliminate (100% reduction) episodes of AFib greater than 24 hours or greater than about 5.5 hours (e.g., greater than 5.5 hours, greater than 5 hours, etc.).

The reduction may be evaluated following a comparison period as described herein. For example, a reduction may be evaluated after at least 7 days post-start of pharmacotherapy or at least about 14 days post-start of pharmacotherapy. For example, the reduction may be evaluated over any evaluation period during treatment at a given dosage such as an evaluation period of days 1-7 after starting pharmacotherapy, days 8-14 days after starting pharmacotherapy, etc., for a 7-day evaluation monitoring period.

A patient is “responsive” to pharmacotherapy (e.g., budiodarone therapy as disclosed herein) if the patient is currently capable of evidencing an efficacious result when administered an approved dosage of budiodarone. In one preferred embodiment, prior to initiating treatment of the patient using the methods described herein, the qualified patient is further evaluated to determine that the patient is suitable to initiate said therapy. The term “a suitable patient” is one who either is currently being treated with a blood thinner or is a patient determined as not having a blood clot. As used herein, a “suitable patient” or “patient” is one who has been experiencing paroxysmal or persistent AFib for about 6 hours or less, or is a patient who has been experiencing paroxysmal or persistent AFib but has been determined to not have a blood clot through the use of a transesophageal echocardiogram or other means of confirming the absence of a blood clot in a patient, or the patient is known to have persistent or paroxysmal AFib and is on a blood thinner.

In one embodiment, pharmacotherapy eliminates the number of long duration episodes of AFib lasting over about 5.5 hours or over about 5 hours and/or eliminates the number of long duration episodes of AFib lasting over about 1 hour. In another embodiment, the pharmacotherapy reduces the patient's number of long duration episodes of AFib lasting over about 5.5 hours or over about 5 hours and/or over about 1 hour.

In one embodiment, pharmacotherapy reduces the symptoms associated with AFib in a symptomatic patient by at least about 15% and preferably at least about 25%, and more preferably by at least 40% when measured after at least 7 days post-start of pharmacotherapy and preferably at least about 14 days post-start of pharmacotherapy.

As used herein, a “non-responsive patient” or patient who is “non-responsive to pharmacotherapy therapy” is a patient that has been treated with pharmacotherapy and does not meet evidence of an efficacious result, as defined above, at any approved dose of the drug. In such cases, the non-responsive patient is removed from pharmacotherapy. When evaluated by the attending clinician, such non-responsive patients may be referred to ablation for the treatment of their AFib.

In one embodiment, those patients qualified for pharmacotherapy have been identified as being refractory to at least one other method of treatment for AFib including ablation.

In one embodiment, those patients qualified to initiate pharmacotherapy are restricted to AFib symptomatic patients.

As used herein, “other methods to treat AFib” may comprise ablation, administering one or more blood thinners and/or other clot prevention measures, heart rate control measures, and/or heart rhythm control measures. Examples of such methods to treat AFib may include administering a beta-blocker (e.g., atenolol, bisoprolol, carvedilol, metoprolol, propranolol, timolol), a calcium channel blockers (verapamil, diltiazem), a blood thinner (warfarin, coumarin, Jantoven, aspirin, apixaban, dabigatran, enoxaparin, heparin, rivaroxaban), a sodium channel blocker (e.g., flecainide, propafenone, quinidine), and/or a potassium channel blockers (e.g., amiodarone, dofetilide, sotalol).

A patient may be deemed “refractory to one or more prior methods to treat their AFib” or “refractory to one or more other methods to treat their AFib” if they have been treated with the one or more prior and/or other methods to treat AFib (e.g., the other methods to treat AFib, as defined above), and their AFib has been deemed unresponsive and/or insufficiently responsive, and/or of decreasing responsiveness to the one or more methods. The “one or more prior therapies” may be ongoing and/or may have been terminated.

As used herein, the term “smart” refers to the computational capabilities of a device. The computational abilities of “smart devices” (e.g., smartphone or tablet) discussed herein may allow for user interaction (e.g., via a touchscreen) and/or for running applications on the smart device.

As used herein, the term “an AFib burden of at least 2.5%” means that in the absence of therapy according to the present disclosure (e.g., in the absence of treatment with budiodarone and/or other heart rhythm drugs that attenuate the extent and/or duration of episodes of AFib and/or symptoms associated with AFib), the patient has episodes of AFib where the aggregate of the duration of each episode over a set period of time is at least 2.5% of the total amount of time in said set period. So, for a patient monitored for 20 days (or 480 hours), at least 2.5% AFib burden means that the total (cumulative) period of time where the patient experiences one or more episodes of AFib is at least 12 hours (2.5% of 480 hours). This AFib burden may be independent of the number, if any, of episodes of long duration AFib. As such, the one or more episodes of AFib that in the aggregate total at least 12 hours can be a single episode or many episodes of less than 1 hour each.

As used herein, the term “budiodarone” refers to (S)-sec-butyl2-(3-(4-(2-(diethyl amino)ethoxy)-3,5-diiodobenzoyl)benzofuran-2-yl)acetate as well as pharmaceutically acceptable salts thereof. Budiodarone, as the free base, is represented by the following formula:

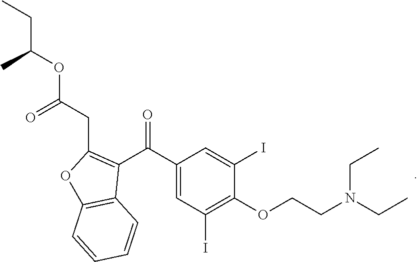 [SMILES: CC[C@H](C)OC(=O)Cc1oc2ccccc2c1C(=O)c1cc(I)c(OCCN(CC)CC)c(I)c1]

Pharmaceutically acceptable salts of the free base are included in the term “budiodarone”) and include all approved pharmaceutically acceptable salts. Inclusive of acceptable salts of budiodarone are budiodarone tartrate or budiodarone citrate. In an embodiment, the pharmaceutically acceptable salt may be a polycarboxylic salt. As is appreciated in the art, the salt disassociates from the free base in vivo. Accordingly, in calculating the serum blood level of budiodarone, the molecular weight of the free base is used to determine molarity. In addition, when a salt other than the tartrate salt is administered, the dose of budiodarone tartrate salt that is used herein will necessarily be changed to reflect the molecular weight change due to the different salt.

The term “wearable” refers to any device that can be worn by a user, e.g., as an accessory, as clothing and/or embedded in clothing, etc., or that can be subdermally implanted in a patient.

As used herein, a patient is “fitted with a wearable” when the wearable is being worn by, is subdermally implanted into, or is otherwise fixed to, the patient to be able to measure the heart rhythm of the patient.

### Instrumentation

The instrumentation used in the methods described herein is designed to be fitted to the patient as part of either the qualification protocol and/or the pharmacotherapy used with the patient as described herein.

Wearables that are used for diagnosis are conventional and aim to inform the clinician whether the patient either has AFib or not and/or if the patient has AFib, possibly information about the patient's AFib burden and the number of episodes of AFib. Such diagnostic analysis fails to address any pharmacotherapeutic suitability, efficacy, and/or dose, which extends beyond diagnosis. The pharmacotherapeutic methods described herein include identifying patients who qualify for drug therapy and/or monitoring the qualified patient during treatment with the pharmacotherapy to assess the drug's effectiveness (e.g., in reducing the number of episodes of long duration AFib). The monitoring may allow for dose adjusting for the patient until the patient is deemed to be responsive to therapy or is disqualified from pharmacotherapy due to a failure to respond. The pharmacotherapeutic methods described herein allow for continuous monitoring of the patient to determine an efficacious dose of the drug as well as to ensure that the patient remains responsive to that dose. Accordingly, the wearables described herein are designed and/or selected to be robust for extended use, and/or comfortable and/or easy to use by the patient.

In practice, the wearable may include a cardiac monitoring component, which may be either an assisted or an unassisted component, for measuring heart rhythm in patients. Many of the wearables are engageable and removable by the patient or are sub-dermally implanted. The specific cardiac monitoring component employed in the wearable is not critical, provided it can accurately measure the heart rhythm. The wearable may be capable of reporting measured heart rhythm data, such as by generating and/or transmitting data indicating the length of time of a detected period of AFib, and/or by generating and/or transmitting data indicating the number of episodes of long duration AFib detected (e.g., over a time period of interest, such as a week, a month, etc.). Also, or alternatively, the wearable may be configured to transmit heart rhythm data to a device configured to detect AFib and/or determine the length of time of a detected period of AFib and/or a number of episodes of long duration AFib (e.g., over the time period of interest). The wearable is approved by one or more regulatory bodies, such as the US Food & Drug Administration (FDA).

### Assisted Components

An assisted cardiac monitoring component may use photoplethysmography (PPG) to detect a patient's heart rate and rhythm. PPG is a conventional technology found in standard oximeters, measures light reflection in tissue to detect arterial pulsations and, accordingly, heart rhythm patterns. However, to continuously and/or semi-continuously measure heart rhythm, one must account for the fact that PPG signals generated during patient movement are often distorted, weak, and have a significant amount of noise relative to detected data. To account for such deficiencies, an algorithm may be used to reduce enough the distortion and/or noise to provide a reliable signal. In one embodiment, both a PPG sensor and accelerometer are employed with an algorithm that allows for appropriate (e.g., sufficient signal-to-noise ratio). When so assisted, PPG may allow for reliable detection of both heart rates and heart rhythm. See, for example, Wojcikowski, et al., Photoplethysmographic time-domain heart rate measurement algorithm for resource-constrained wearable devices and its implementation, SENSORS 20, no. 6 (2020): 1783 which is incorporated herein by reference in its entirety.

In some embodiments, the cardiac monitor component uses piezoelectric material and/or rhythm electroactive polymers to detect blood flow, thereby indirectly measuring heart rhythm.

In some embodiments, a combination of PPG and/or piezoelectric measurements and electrocardiogram data from single-electrode wearables (i.e., iEKG, e.g., as opposed to EKG, which refers to a conventional multiple-electrode electrocardiogram) can be combined to increase the specificity of the measurement. The iEKG (i.e., iECG) and the PPG or the piezoelectric data can originate either from two separate devices communicating by transceivers, for example, an armband and a smartwatch, or they can originate from a single device, for example, a wristband on a smartwatch (e.g., the Kardia™ Band on an iWatch). Once an arrhythmia is detected in the PPG data, the corresponding (e.g., in time) iECG data may be analyzed by an algorithm.

### Unassisted Components

In another embodiment, the wearable may include an unassisted cardiac monitor component, such as a portable electrocardiogram. The portable electrocardiogram component may be wearable, engageable at-will by the patient, and/or capable of transmitting data, e.g., via a built-in antenna or Bluetooth data transmitting device. The wearable comprising the unassisted cardiac monitor component can measure heart rates and heart rhythms. The wearable may be configured to detect and log AFib burden and/or long duration (episode) of AFib (LEAF) over an observation period (e.g., of about 2 weeks or longer). The unassisted cardiac monitor component employs direct measurement, which means that the device is reading electrical signals generated by the heart. The direct measurement may be less affected by noise and distortion than indirect measurements, such as PPG measurements, which may enable transmitting measurement data to a clinician without the use of an algorithm, and/or with reduced use of any algorithm or data processing.

In some embodiments, the wearable may further include a specialized accessory, such as a cardiac monitor device, that can detect the electrical activities of a heart including heart rhythm through an electrode. The specialized accessory may be capable of initiating transmission and/or may comprise and/or be connected to a transmission device.

In some embodiments, the cardiac monitor device can be a component that is an integral part of a single wearable device, such as a smartwatch. By providing a single device that is wearable by a user and is capable of monitoring the electric field of the heart of the user, the electrical activities of the heart can be monitored continuously over a prolonged period, such as days or even months.

In some embodiments, the cardiac monitor device may include an analog-to-digital converter capable of digitalizing measured electric field data (e.g., measured potential difference data) to transmit and/or store in memory the measured data as digitized data. The cardiac monitor device can include an output that can transmit signals carrying information about the difference of potential between the limb and the body to an external circuit. The output can take various forms. In one case, the output can be a transceiver that communicates to another transceiver/receiver in another unit, for example, a watch or a tablet. In one embodiment, a central analysis center (e.g., a remote lab, such as a CORE lab and/or a remote data analysis center and/or a server) may interpret and/or summarize the AFib data and/or the LEAF data. The central analysis center may also transmit a dose adjustment recommendation to the treating clinician, without a need for the patient to visit his or her physician (e.g., as part of a patient monitoring program). Also, or alternatively, an artificial intelligence algorithm may be used to determine the dose adjustment and may transmit the dose adjustment recommendation to the physician.

### Further Aspects

In one embodiment, the wearable may be a small consumer electronic device, for example, a watch, an armband, a ring, a strap, and/or a wristband. The wearable may include a housing that carries the cardiac monitor component and any associated circuitry, CPU, and the like. The wearable can also be worn at other locations on the user, including, but not limited to, the wrist, leg, neck, and/or body. The wearable may comprise a specialized accessory capable of communicating with another electronic device such as a tablet, a laptop computer, a desktop computer, and/or other similar devices, which, in turn, can communicate to a cloud network to transmit information from the device to the clinician. Also, or alternatively, the specialized accessory may be capable of transmitting information directly and/or via a network to the clinician.

The heart rhythm and iECG wearables may be Bluetooth, Z-Wave, Zigbee, and/or Advanced and Adaptive Network Technology (ANT)-enabled. For example, the iECG and/or the heart rhythm monitoring and/or recording device (e.g., PPG or a piezoelectric heart rhythm monitoring and/or recording device) may be paired with an application that may be configured to automatically detect AFib based on data from the iECG and/or the heart rhythm monitoring and/or recording device. The iECG device and/or the rhythm monitoring and/or recording device may be configured to transmit data to the application. The data can be transmitted using one or more data transmissions methods such as Bluetooth, Z-Wave, Zigbee, or ANT protocols. The application may be configured to analyze the data using a proprietary software. Based on the data from the iECG and/or the heart rhythm monitoring and/or recording device, the application may be able to interpret and/or detect AFib with a sensitivity of >90% and a specificity of >80%.

Patients 1 with a wearable as described herein can use the wearable to record heart rhythm and detect AFib. The recording can be continuous and/or semi-continuous. The specifics of the recording can be programmed into the device, directly or indirectly (e.g., remotely). AFib data may be stored securely, such as in a cloud-based data repository using highly secured protocols.

Examples of commercially available assisted and unassisted wearables include, without limitation, the following:
MCOT® wearables sold by Philips Biosciences, Best, Netherlands. This device is a wearable patch that can send iECG data automatically via a wireless connection to a central analysis center (e.g., a CORE lab). This system is configured to provide data sufficient to enable a clinician to determine if dose escalation and/or drug discontinuation is warranted.
An ePatch extended wear Holter monitoring system sold by Philips Biosciences, Best, Netherlands. This device is configured to record data indicating AFib and/or long duration (episode) of AFib (LEAF). This device is configured to record and/or store iECG data continuously. This data is then archived and analyzed centrally.
   There are many other wearables, some of which are FDA approved, which can be used in place of those recited above including, by way of example only, Zio by iRhythmtech, San Francisco, California, USA; Frontier X2, Fourth Frontier, Austin, Texas, USA, wearables by VivaLink, Campbell, California, USA, just to name a few. The above and other wearables discussed herein represent a non-exhaustive list of wearables suitable for use in the methods described herein. The specific wearable to be used is not critical, as long as it is capable of measuring heart rhythm (e.g., generating a signal and/or data from which heart rhythm can be determined). The wearable used should be capable of measuring the heart rhythm of a patient fitted with the wearable continuously or semi-continuously for a required time period as described herein (e.g. a required time period for determining a baseline, for monitoring during pharmacotherapy, etc.). Further, the wearable should be small enough and/or comfortable enough for a patient to wear and/or be fitted with the wearable for the required time period.

### Methodology

The disclosed methods allow the attending clinician to identify and/or treat a qualified patient with budiodarone, as well as to assess the efficacy of the therapy based on heart rhythm data monitored over a period of time. The period of time may be an extended period of time, which may be measured in weeks, months, and/or years. The monitoring may comprise monitoring heart rhythm data and/or using said heart rhythm data to determine a condition of the patient's AFib, which may be determined based on one or more of the patient's AFib burden or the number or duration of AFib episodes over the period of time (e.g., a frequency of AFib episodes and/or long duration AFib episodes) or symptoms associated with AFib. This represents a new paradigm in treating AFib, as it allows the clinician to do one or more of the following:
assess the extent of the disease in the patient;
determine if the patient qualifies for pharmacotherapy;
evaluate the efficacy of the therapy, in the short term (1-6 months) and/or in the long term (after 6 months);
dose adjust the patient to achieve and/or maintain a therapeutic result; and/or
remove patients from pharmacotherapy if they are non-responsive to the therapy.

In contrast, conventional monitoring of a patient is typically diagnostic in nature, e.g., conducted during a single short-duration period typically of no more than two-weeks using a Holter monitor or the equivalent. Such diagnostic methods allow the clinician to at best confirm the presence of AFib. In most cases, the clinician will then place the patient on blood thinners. Alternatively, the clinician can place a diagnosed patient on pharmacotherapy, which might involve heart rate reduction using beta-blockers and/or calcium channel blockers. Regardless, once diagnosed, conventional monitoring is typically terminated.

In cases where a clinical evaluation of AFib is done for clinical investigative purposes, it is common to use an implantable (invasive) device that requires surgical insertion and, subsequently, surgical removal. Such investigations are typically done to understand the underlying risks of stroke and congestive heart failure relative to either the AFib burden and/or AFib duration. See, for example, Turakhia M. P. et al., Circ. Arrhythm. Electrophysiol., 2015, 8(5):1040-7. In some cases, non-invasive means to monitor the patients were sometimes used but typically the monitoring is of short duration. See, e.g., Go, et al., JAMA Cardiology, 2018, 3(7):601-608. Regardless, such clinical evaluations failed to address or provide a method for efficaciously delivering budiodarone to a patient and then evaluating the effect of this drug on the patient's AFib burden or episodes of long duration AFib over extended periods of time with the option to dose adjust patients to achieve the desired therapeutic result.

Unlike prior protocols for treating AFib using drugs that limited the patient's heart rate, the methods described herein are directed at treating the patient's heart rhythm to reduce the causative reasons for stroke and heart failure and to alleviate or eliminate symptoms associated with AFib. By coupling the ability to continuously or semi-continuously monitor the heart rhythm with suitable drugs that limit the number and extent of long duration episodes of AFib, the clinician can significantly reduce the risk of stroke or congestive heart failure. Still further, by monitoring the patient after initiation of drug therapy, the clinician can now evaluate the efficacy of the drug, adjust the dose as needed to enhance overall efficacy or identify those patients who are non-responders to such therapy who are removed from the therapy.

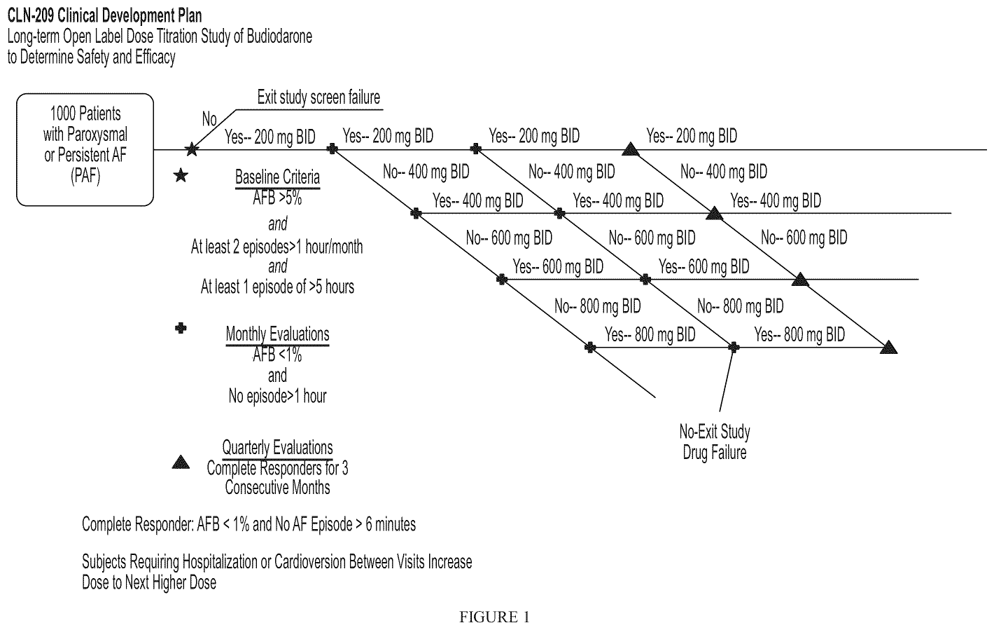

One embodiment of this approach is depicted in FIG. 1, which schematizes an iterative process to evaluate a patient, qualify a patient for budiodarone therapy, and treat the qualified patient. In FIG. 1, an exemplary cohort of patients, who have been diagnosed with either paroxysmal and/or persistent AFib, are evaluated for their AFib burden. Those evidencing a suitable AFib burden (“Yes” at star decision point), in this case, 5% or more, but this AFib burden may be a different threshold, such as 2.5%, and/or a patient and/or cohort-specific threshold, may be selected to continue in the qualification process, while those who fail to meet this threshold are excluded (“No” at star decision point). The patients may also be evaluated for the duration of their AFib episodes. In this particular embodiment, patients are further qualified for budiodarone therapy (“Yes” at star decision point) if there is evidence of at least 1 episode of AFib lasting more than about 5 or about 5.5 hours over a 1-month period and/or two or more episodes of AFib lasting more than about 1 hour over a 1-month period. Patients who fail to meet either of these criteria are excluded (“No” at star decision point) as presumably having a very low risk of stroke or congestive heart failure. Qualified patients are placed on an ascending dose regimen of budiodarone, which has been shown to significantly reduce episodes of AFib, including episodes of long duration AFib, which, by definition, reduce AFib burden. Other drugs that behave similarly to budiodarone may be used in place of and/or in addition to budiodarone in the methods described herein.

In the iterative process of FIG. 1, an ascending regimen is employed to assess whether budiodarone is effective in treating long duration episodes of AFib and/or at what dose(s). Given that the AFib burden and/or the number and duration of episodes of long duration AFib vary from patient to patient, different doses of budiodarone may be effective for different patients. However, heretofore, a clinician prescribing budiodarone at a first dose was blinded from whether that dose was efficacious. As per FIG. 1 and the Examples below, the methods described herein allow the clinician to assess efficacy at a first dose and/or to dose adjust in an ascending and/or descending protocol in an iterative process until either a therapeutic result is achieved, and/or the patient is deemed to be a non-responder. As to such non-responders, they are removed from budiodarone therapy.

In an ascending regimen, patients are first administered a low dose and may have the dose increased in case the patient is non-responsive and/or insufficiently responsive (e.g., “No” at the plus signs). A patient is determined non-responsive if they do not demonstrate evidence of drug efficacy at the maximum dosage. A descending regimen can also, or alternatively, be employed, in which a highest dose of budiodarone is administered to the patient, and efficacy is determined for decreasing dosages. One advantage of a descending protocol is that non-responders could be determined in the first iteration and not the last. However, an ascending protocol may have the benefits of first finding a lowest effective dosage and/or reducing the risk of side effects. A combination of an ascending dose regimen and a descending dose regimen can be used to fine-tune the minimum effective dose of budiodarone in a patient. For example, if a 200 mg twice-a-day dose regimen of budiodarone is ineffective and 400 mg twice a day is efficacious, the attending clinician could modify that dose in a descending manner to determine if 350 mg twice a day or 300 mg twice a day of budiodarone would be efficacious. Such has particular benefits if the patient evidences side effects of budiodarone therapy at 400 mg twice a day but not (or not as much) at a lower but still efficacious dose of 350 mg or 300 mg twice a day.

In one embodiment, treatment of a patient with budiodarone may result in reducing the number and/or frequency of episodes of long duration AFib and/or may result in reducing the AFib burden. This is in contrast to drugs that reduce heart rate but have little to no ability to reduce the duration of AFib episodes and/or the AFib burden. AFib burden and episodes of long duration AFib are distinct. As to AFib burden, the number and duration of each AFib episode in a patient are measured to determine an AFib burden. A patient having only very short-duration episodes of AFib may still be assigned a higher AFib burden than a patient with infrequent episodes of long duration AFib. Hence, a patient who has 8 episodes of AFib each having a duration of 45 minutes in a given 24-hour period would be assigned an AFib burden of 25% (6 hours/24 hours). In contrast thereto, a patient with a single episode of AFib lasting for 5 hours during a 24-hour period would be assigned an AFib burden of 20.8%. However, the latter patient with the single AFib episode may be at greater risk of a stroke than the former patient. Accordingly, patients having an AFib burden of less than 2.5% are very unlikely to have long duration AFib and, accordingly, may be determined not qualified for treatment with budiodarone.

Still further, transitioning a patient from either paroxysmal AFib to persistent AFib or from persistent AFib to permanent AFib may be unwanted disease progression. By reducing the extent of long duration AFib and the number of AFib episodes, treatment of a patient with budiodarone, as disclosed herein, can maintain the patient in their current stage of the disease (and may see regression from persistent to paroxysmal AFib), at least for a longer period of time than without budiodarone treatment, thereby inhibiting the progression of the disease.

In comparison, treatment of AFib with blood thinners does not address the cause of AFib and does not reduce the underlying concerns (e.g., increased stroke risk, increased CHF risk) with either AFib burden and/or with medium-long duration and longer-duration episodes of AFib. Moreover, using blood thinners does not prevent or slow a patient from progressing from paroxysmal AFib to persistent AFib or from persistent AFib to permanent AFib. Still, further, the use of blood thinners introduces another set of issues with bleeding. For example, as discussed previously, about 6% of patients on blood thinners may be expected to experience an adverse event due to the administration of the blood thinner requiring an emergency department visit, with about 3% requiring a subsequent hospital stay. Most adverse events caused by blood thinners are bleeding events, with catastrophic bleeds requiring hospitalization. A catastrophic bleed may include bleeding into the abdominal cavity (requiring surgery for recovery) or intracranial bleeding (deadly). Moreover, blood thinners reduce the risk of stroke, but do address the underlying reasons that long duration episodes of AFib cause or increase risk of strokes in patients with AFib.

Accordingly, in one embodiment, patients who are on blood thinners for treatment of AFib and show efficacy of budiodarone therapy (e.g., as described herein) may be identified for budiodarone therapy with monitoring, e.g., as described herein. In an embodiment, if the budiodarone therapy is found to sufficiently reduce a risk of stroke in the patient (e.g., by reducing one or more of the AFib episode duration and/or number and/or AFib burden a desired amount and/or an amount associated with reduced risk of stroke and/or CHF), the patient may be removed from blood thinners (e.g., by the clinician). Since budiodarone does not pose a risk of the adverse effects of blood thinners (e.g., bleeds), if the budiodarone therapy is found to sufficiently reduce risk of stroke in a patient to be comparable to a risk of stroke of the patient on blood thinners or to be comparable with a combined risk of a stroke and bleeds in the patient on blood thinners, the patient blood thinners may be placed on budiodarone therapy and removed from blood thinners. For example, if the budiodarone therapy is found to reduce the duration of AFib episodes for a patient to less than 1-hour duration episodes of AFib (e.g., as a maximum length episode over an evaluation period, on average duration value over an evaluation period, etc.), the patient may be removed from blood thinners (e.g., by the clinician). The risk of stroke in the patient may be determined based on individual characteristics and risk factors for the patient, such characteristics captured by the patient's CHA₂DS₂-VASc scores (2 or less). as paroxysmal AFib and persistent AFib with low CHA₂DS₂-VASc scores (2 or less) and high CHA₂DS₂-VASc scores (3 or higher). Thus, patients with different CHA₂DS₂-VASc scores may require different levels of reduction in their AFib to be removed from the blood thinner. For example, a patient with a low CHA₂DS₂-VASc score (e.g., 2 or less) may be removed from blood thinner if budiodarone therapy is found to reduce or eliminate episodes of AFib greater than about 5.5 hours in duration or greater than 5 hours in duration, or reduce or eliminate episodes of AFib greater than 1 hour in duration, and/or if the budiodarone therapy is found to reduce an AFib burden to below 5%, below 2.5% over a comparison period, etc. A patient with high CHA₂DS₂-VASc score (e.g., 3 or more), may require a greater reduction in AFib level to be removed from blood thinners. For example, a patient with a high CHA₂DS₂-VASc score may be removed from blood thinner if budiodarone therapy is found to reduce or eliminate episodes of AFib greater than 1 hour in duration or greater than 0.5 hours in duration or greater than 0.1 hours in duration, and/or if the budiodarone therapy is found to reduce an AFib burden to below 1% over a comparison period, etc. In an example, a patient with a very high CHA₂DS₂-VASc score (e.g., 6 or more), may be removed from blood thinners only with the elimination of episodes of AFib greater than 0.1 hours in duration, or may not be removed from blood thinners.

In view of the above, according to the present disclosure, a patient's heart rhythm may be monitored over an extended period to assess the efficacy of a drug that is intended to control such long duration episodes of AFib. Devices such as a Holter monitor can be used to monitor a patient's heart rhythm over a short period of time (e.g., 1 day, 2 days, or 3 days and even up to 14 days), but such monitoring cannot provide a comprehensive and ongoing analysis of the patient's AFib and/or how a drug treatment is impacting the number and duration of episodes of AFib over extended periods of time (e.g., of months or more). Such short-duration analysis provides only a diagnostic analysis, not a therapeutic analysis, and fails to provide a full picture of the heart health of the patient. Due to the sporadic and dynamic nature of AFib (paroxysmal and persistent), and variability across patients, an accurate measurement of a level of extent of AFib in a patient and the benefits of pharmacotherapy in treating AFib can only be obtained by monitoring over prolonged periods of time, such as longer than 1 month, longer than 3 months, etc.

### Drug Therapy

There are many conditions where the attending clinician can evaluate a patient based on a static number that represents a meaningful long-term average. For example, in diabetes, the three-month value for hemoglobin A1C levels (also referred to as HbA1C test is a blood test) provides an excellent indicator for the average daily blood glucose levels. In addition, liver function, prostate health, thyroid health, etc. can all be evaluated based on a specific number that provides meaningful information to the clinician.

The clinicians treating paroxysmal and/or persistent AFib have had to rely upon brief/intermittent monitoring of the patient's heart rhythm, such as via an EKG (electrocardiogram) and/or a Holter monitor. However, such brief monitoring may miss critical data points that could only be obtained by monitoring over longer periods of time. This may cause a clinician to avoid pharmacotherapy without a means to monitor the drug's effectiveness in the patient. Rather, a patient diagnosed with AFib may be placed on symptom and/or risk reduction treatments, such as blood thinners. However, while blood thinners will reduce the risk of clot-related strokes (e.g., clots arising from AFib), there is a corresponding increased risk of uncontrolled bleeding caused by the blood thinners which can lead to death.

Pharmaceuticals, such as sotalol, as well as beta-blockers and calcium channel blockers, have been used to treat AFib. Examples of calcium channel blockers include but are not limited to amlodipine, diltiazem, felodipine, isradipine, nicardipine, nifedipine, nisoldipine, and verapamil. Examples of beta blockers include but are not limited to acebutolol, atenolol, bisoprolol, metoprolol, nadolol, nebivolol, and propranolol. Drugs with combined modes of action, such as amiodarone, also exist. However, each of these pharmaceuticals is currently deemed inferior pharmacotherapies for the treatment of AFib, as they either do not provide sufficient control of the heart rhythm or they produce serious side effects in the patient (e.g., amiodarone has a long half-life and its accumulation after chronic use is associated with a wide variety of toxicities including lung, liver, and ocular effects; its use requires close supervision of recipients to detect early signs of irreversible and potentially fatal complications [see, e.g., Wolkove N, Baltzan M., “Amiodarone pulmonary toxicity,” Canadian Respiratory Journal 16(2): 43-8, 2009]).

Budiodarone, a drug with a combined mode of action, has been shown to reduce episodes of long duration AFib without a significant increase in the QT interval. Budiodarone has a pattern of cardiac ion channel effects that are like amiodarone by design with enhanced late Na⁺ channel blockade. Budiodarone's deliberately altered metabolism results in a much shorter half-life than amiodarone, however, enabling the avoidance of accumulation-related toxicities seen with amiodarone and complete inactivation and elimination from the body within hours to days of discontinuation. The shortened half-life allows for rapid achievement of steady-state blood levels within 2-3 days of commencing the drug and the ability to adjust the dose if necessary to establish the minimally effective dose for each subject provided there is real-time information available on efficacy (e.g., changes and/or reduction in AFib burden and/or long duration episodes of AFib). Indeed, budiodarone represents a significant advancement in treatment for AFib, since episodes of long duration AFib are substantially the cause of clot formation that may lead to strokes and/or congestive heart failure. However, AFib is a variable disease, with some patients requiring different dosing of a drug to achieve desired results. As noted earlier, the inability to monitor patients in a manner that could evaluate the effectiveness of the drug has been a major drawback to pharmaceutical intervention. This has led to clinicians opting for the use of blood thinners and/or risk reduction measures, instead of proactive pharmaceutical intervention.

The wearables and methods described herein provide sufficient data to the clinician on an ongoing basis to determine the long-term effectiveness of drugs in treating paroxysmal and persistent AFib. As such, methods that integrate data generated by wearables with a drug that can control long duration AFib and/or reduce AFib burden may allow for more effective treatment of AFib, which is a long-felt need in the art. Moreover, the ability to determine the long-term effectiveness of a drug may allow for dose-adjusting the patient to effectively achieve control over episodes of long duration of AFib.

### Treatment Regimens Using Budiodarone

Budiodarone has been shown to reduce episodes of long duration AFib in patients thereby reducing the risk of stroke and congestive heart failure. However, to effect therapy over a broad spectrum of patients having varying frequencies in the number and duration of episodes of long duration of AFib using budiodarone requires that the clinician monitor each patient for efficacy at a given dose. Such represents a personalized medicine approach to a disease that is treatable by budiodarone.

If a patient is non-responsive at that dose, an increase in dosing is warranted and the process is repeated until it is determined that the patient is responsive at a given higher dose or is non-responsive. Patients who are non-responsive at all tested doses are removed from budiodarone therapy.

Still further, patients who have either paroxysmal or persistent AFib and, who are refractory to one or more prior methods for treating their disease, may be candidates for the methods and procedures described herein. Such patients may be screened (qualified) for treatment by determining their baseline AFib burden and/or the number and extent of their long duration AFib episodes. Their baseline may also be used to assess the relative reduction in and/or the complete elimination of these symptoms based on budiodarone therapy and/or to identify patients as non-responders to budiodarone therapy. Accordingly, a method to treat patients with paroxysmal or persistent AFib, wherein said patients are refractory to one or more prior therapies to treat their AFib, may comprise:
for a patient who is refractory to the one or more prior methods to treat AFib and who is fitted with a wearable that monitors heart rhythm data and is capable of transmitting said data;
a) identifying the number of episodes of long duration AFib and the extent of AFib burden in said patient, wherein the identified number and duration satisfy criteria to qualify the patient for treatment with budiodarone by a clinician;
b) administering budiodarone to said patient at the lowest approved therapeutic dose;
c) monitoring said patient with a wearable for the efficacy of budiodarone administered at said lowest therapeutic dose to assess whether said dose is efficacious for said patient wherein said monitoring for efficacy is initiated at least about 7 days or at least about 2 weeks after the start of said therapy;
d) for patients who did not achieve an efficacious result at the lowest possible dose of said pharmaceutical at the end of the second period of time, dose adjust the amount of said pharmaceutical one or more times as necessary to achieve an efficacious result using at least the length of the second period of time to assess efficacy, or if an efficacious result is not achieved when evaluated at the maximum approved dose, remove said patients from said therapy; and
e) for patients who did achieve efficacious results, continue to monitor their heart rhythm to ensure that the dose of said pharmaceutical remains efficacious provided that if said dose is no longer efficacious then repeating the steps of c and d) as needed.

Because of the lack of sufficient information to ascertain the efficacy of a given dose of any drug prescribed to reduce the number of episodes of long duration AFib in the treated patient, a clinician would invariably avoid doing so particularly given the variability in patients as well as in the disease itself. Still further, many clinicians view that treating (reducing) the heart rate is an appropriate method for treating AFib optionally coupled with blood thinners. See, for example, Atrial Fibrillation—Treatment, https://www.nhs.uk/conditions/atrial-fibrillation/treatment/ where, in 2021, the authors were controlling heart rates as part of an appropriate approach to treating AFib.

Now, as per the methods described herein, a clinician can evaluate a patient's heart rhythm data over an extended period of time such as over at least about 3 months, over at least about 6 months, over at least about 12 months, and potentially for the remainder of the patient's life, to determine the number and/or duration of episodes of long duration AFib and whether budiodarone therapy is and remains efficacious.

In one embodiment, which is illustrated in the Examples and FIG. 1, for example, the clinician may initiate dosing of budiodarone at a minimal level of about 200 mg twice a day (bid) and then assess whether that dose is effective in eliminating episodes of long duration AFib. The clinician can review data at the initial dosing level and dose-adjust the patient one or more times, e.g., as necessary to arrive at a dose where the patient is substantially free of and/or is free of episodes of long duration AFib. In general, the incremental increases in dosing can be about 200 mg twice a day. So, in this approach, the dosing can be changed from about 200 mg bid, to about 400 mg bid, or to about 600 mg bid, and so on up to about 800 mg bid and possibly higher if the attending clinician determines that there is a benefit to higher doses. Also, or alternatively, though not shown in FIG. 1, smaller or larger increments of dose adjustments (e.g., +/−100 mg bid or +/−300 mg bid, etc.) may be chosen to fine-tune the dosage to the patient, and the clinician may lower a dosage that achieves sufficiently efficacious results so long as the lowered dosage also achieves sufficiently efficacious results as described herein.

### Systems

The methods described herein can be conducted either in a unitary device, such as a wearable that can record, store and transmit the heart rhythm data. However, systems of multiple devices can also be used, provided that such systems include:
a) a wearable that collects heart rhythm data from a patient diagnosed with either paroxysmal atrial fibrillation and/or persistent atrial fibrillation;
b) a CPU, configured to communicate with and/or receive the heart rhythm data or data based on the heart rhythm data from the wearable, and that collects and stores the heart rhythm data and/or data based on the heart rhythm data; and
c) a transmission component (e.g., device) that is part of and/or communicates with the CPU (chest pain unit) and that is configured to send the data and/or information based on the data (such as alerts or alarms, analyzed AFib episode numbers, etc.) directly and/or indirectly to one or more designated recipients (e.g., the patient, one or more clinicians, etc.).

In one embodiment, the CPU is capable of evaluating the stored and/or received data to determine the presence and number of medium-long and/or longer-duration episodes of AFib. Evaluation by the CPU can be continuous or periodic. The CPU can be programmed to alert the clinician and/or patient and/or a central analysis center (e.g., remote lab and/or analysis center) of medium-long and/or longer-duration episodes of AFib deemed dangerous for a patient (e.g., in a number above a threshold number or of a duration above a threshold duration). The wearable may also or alternatively comprise a storage unit for storing collected data and the storage unit can be programmed to evaluate the stored data and alert the clinician and/or the patient and/or the central analysis center of any potentially harmful heart rhythm irregularities. The collected and/or evaluated data may be continuously or periodically reviewed by the clinician or health care professional (e.g., may be pushed to a computing device accessible by the clinician continuously or periodically, and/or pulled based on a request by the computing device accessible by the clinician). Periodic evaluation can be, for example, once a day, once a week, twice a month, once a month, etc. Also, or alternatively, the collected and/or evaluated data (at the wearable, the CPU, the central analysis center, or at another computing device accessible to the patient and/or clinician) may be filtered for events deemed dangerous for the patient (e.g., AFib episodes in a number above a threshold number or of a time duration above a threshold duration, an AFib burden above a threshold level), and if such an event is detected, an alarm (or alert) may be triggered for the patient and/or sent to the clinician (e.g., by the wearable, by the CPU, by a computing device accessible to the clinician, at the central analysis center, etc.). Use of an alert (alarm) is optionally available and can be provided immediately after the detection of a long duration AFib episode to provide a clinician and/or the patient with knowledge of the event. The comparison period is independent of the alert and provides data that allows the clinician to compare the efficacy of the drug over time against the baseline.

### EXAMPLES

In the examples that follow, and throughout the specification, the following abbreviations have the following meanings. If an abbreviation is undefined, then it has its conventional medical meaning.
AFib or AF atrial fibrillation
Bid or b.i.d.=twice a day
bpm=beats per minute
hrs=hours
mg=milligrams
PPG=photoplethysmography
SD=standard deviation

Example 1 is provided to illustrate that the use of a wearable can detect AFib episodes during continuous monitoring of a patient.

Examples 2-7 establish the utility of budiodarone in the methods described herein. In these examples, the patients all had surgically implanted pacemakers and as such carried a number of risks associated therewith. See, e.g., Pacemaker, https://www.mayoclinic.org/tests-procedures/pacemaker/about/pac-20384689, (last visited Oct. 26, 2022) which is incorporated herein by reference in its entirety.

### Example 1—Wearables that Detect AFib in Patients on a Continuous Basis

In this example, a male patient suffering from non-permanent AFib was fitted with an MCOT (mobile cardiac outpatient telemetry) wearable device sold by Philips BioSciences, Inc., Best, The Netherlands, and commercially available by prescription in the United States. The MCOT device employs PPG coupled with an algorithm to assess heart rhythm and AFib. The MCOT device is configured to wirelessly and seamlessly provide heart rhythm and AFib data to a dedicated computer.

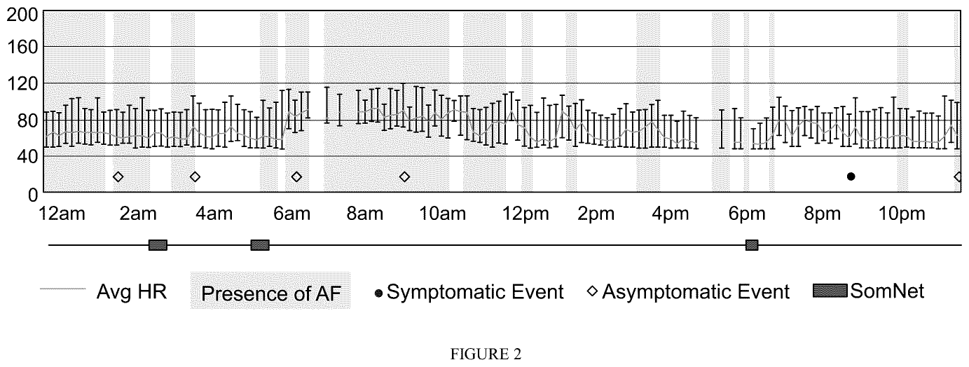

The patient was continuously monitored for 23.5 hrs. During the entire period, the MCOT device measured the patient's heart rhythm and rate. FIG. 2 provides a cardiogram obtained during the monitoring period. Of note is that there were 33 separate episodes of AFib detected and recorded each separated by periods of sinus rhythm or other arrhythmias. The specific details of the analysis of the cardiogram are as follows:

| | |
|---|---|
| Number of AFib Episodes | 33 |
| Longest Period in AFib | 3 hours and 2 minutes |
| Heart Rate (minimum/maximum) | 48/94 beats per minute |
| AFib Burden | 36% |

The above data demonstrates that a wearable as described herein was capable of continuously monitoring the patient's heart rhythm and provided detailed analysis of the number of AFib episodes as well as the extent of AFib burden.

### Example 2—Elimination of Long Duration AFib with Budiodarone

This example is a clinical trial evaluation of budiodarone in treating 6 patients with paroxysmal or persistent AFib who previously evidenced episodes of long duration AFib (greater than 24 hours). In this example, the patients were subject to different conditions (baseline, different dosages of budiodarone, washout), and, for each condition, the individual patient's heart rhythm in the patient cohort was continuously monitored over a two-week period with a pacemaker. The purpose of this example is to establish whether budiodarone reduces long duration AFib lasting more than 24 hours. The different doses and results of this evaluation are provided in Table 1 below:

TABLE 1

| Parameter | | 200 mg | 400 mg | 600 mg | 800 mg | |
|---|---|---|---|---|---|---|
| Mean (SD) | Baseline | Bid | bid | bid | bid | Washout |
| # of AFib episodes | 22(22) | 31 (28) | 37 (54) | 42 (66) | 24 (28) | 37 (49) |
| Avg Duration of | 4.8 (5.2) | 1.7 (2.5) | 0.6 (0.7) | 0.1 (0.2) | 0.5 (0.7) | 2.5 (5.0) |
| episodes | | | | | | |
| Avg. Duration of | 23 (21) | 131 (162) | 48 (57) | 103 (148) | 42 (70) | 40 (43) |
| sinus rhythm | | | | | | |
| Longest AFib | 50 | 7 | 13 | 7 | 5 | 105 |
| Episode - hrs | | | | | | |

The above results demonstrate that budiodarone eliminated over 70% of the episodes of long duration AFib lasting more than 24 hours as compared to a baseline for all budiodarone dose levels with little differences between treatment levels of 200 mg bid, 600 mg bid, and 800 mg bid. In contrast, both baseline and washout results evidence the presence of episodes of long duration AFib lasting more than 24 hours. These results also evidence a significant reduction (more than 64% reduction) in AFib burden (the number of episodes over a 2-week period of time the average duration of the episodes). This reduction correlates well with reducing the risk of a patient transitioning from paroxysmal AFib to persistent AFib or from persistent AFib to permanent AFib.

### Example 3—Elimination of Longer Duration AFib (>24 Hours) During a 12-Week Period

This example is a clinical trial evaluation of budiodarone in treating 6 patients with either paroxysmal or persistent AFib who previously evidenced episodes of long duration AFib (greater than 24 hrs). In this example, each patient in the cohort was continuously monitored for heart rhythm data over a twelve-week period with a pacemaker (surgically invasive procedure) and was treated with different doses (2 weeks each) of budiodarone. In addition, during this trial, the patients were under continuous monitoring and constant clinician supervision.

The purpose of this example is to establish whether budiodarone treatment can reduce longer duration AFib (AFib episodes lasting more than 24 hours) in a clinical study setting and using a pacemaker to evaluate heart rhythm. The results of this analysis are provided in Table 2 below:

TABLE 2

| Episodes of AFib >24 hrs | Number | Duration |
|---|---|---|
| Off Drug (baseline) | 4 | 219 (100%) |
| 200 mg bid | 0 | 0 (0%) |
| 400 mg bid | 0 | 0 (0%) |
| 600 mg bid | 0 | 0 (0%) |
| 800 mg bid | 0 | 0 (0%) |

“Number” indicates the number of longer duration episodes of AFib (i.e., having a duration of greater than 24 hours). “Duration” indicates the amount of time during the 2 weeks spent in the longer duration indicated in hours and a percentage relative to the baseline measurement. As per the above results, 200 mg bid was sufficient to treat all patients in the study by eliminating the number of episodes of AFib lasting over 24 hours. In this case, dose escalation was not required to improve patient efficacy as the 200 mg bid doses of budiodarone were sufficient to eliminate these long duration episodes of AFib.

### Example 4—Reduction of Long Duration AFib (>5 Hours) During a 12-Week Period

This example is a clinical trial evaluation of budiodarone in treating 6 patients with either paroxysmal or persistent AFib who previously evidenced episodes of long duration AFib (greater than 5 hrs). In this example, each patient's heart rhythm was continuously monitored over a twelve-week period with a pacemaker (surgically invasive procedure) and were treated with different doses of budiodarone, each dose being administered for a period of two weeks. In addition, during this trial, the patients were under continuous monitoring and clinician supervision.

The purpose of this example is to establish whether budiodarone reduces long duration AFib lasting more than 5 hours in a clinical study setting using a pacemaker to evaluate the heart rhythm. The results of this analysis are provided in Table 3 below:

TABLE 3

| Episodes of AFib >5 hrs | Number | Duration | |
|---|---|---|---|
| Off Drug (baseline) | 29 | 490 | (100%) |
| 200 mg bid | 3 | 17 | (3.5%) |
| 400 mg bid | 4 | 38 | (7.7%) |
| 600 mg bid | 2 | 14 | (2.8%) |
| 800 mg bid | 1 | 5 | (1%) |

“Number” refers to the number of long duration episodes of AFib having a duration of greater than 5 hours. “Duration” indicates the amount of time during the 2-week period spent in the greater than 5 hour duration AFib, indicated in hours and a percentage relative to the baseline measurement. In this example, all doses of budiodarone provided statistically significant benefits in reducing both the number of AFib events and the duration of AFib episodes over 5 hours, with both the 200 mg bid and the 600 mg bid providing similar results whereas the results for 400 mg bid were exceptionally better than Off Drug condition (baseline data taken in the absence of budiodarone for each patient), but inferior to the 200 mg bid and the 600 mg bid. Finally, the 800 mg bid performed best. At all dosage levels, the reduction in AFib episodes greater than 5 hours was above 85% and the corresponding reduction in AFib burden was above 90%.

In light of the previously discussed relation between long duration AFib episodes and increase the risk of stroke and heart failure (see, e.g., Singer, et al., “Temporal Association Between Episodes of Atrial Fibrillation and Risk of Ischemic Stroke,” JAMA Cardiology, 6(12): 1364-1369 (2021)), the next comparative example measured the reduction in AFib episodes of 1 hour or more in patients.

### Example 5—Reduction of Long Duration AFib (>1 Hours) During a 12-Week Period

This example is a clinical trial evaluation of budiodarone in treating 6 patients with either paroxysmal or persistent AFib who previously evidenced episodes of long duration AFib (greater than 1 hour). In this example, the patients' heart rhythms were continuously monitored over a twelve-week period with a pacemaker (surgically invasive procedure) and were treated with different dosage levels (different doses of budiodarone administered bid) for two weeks at each dosage level. In addition, during this trial, the patients were under continuous monitoring and clinician supervision.

The purpose of this example is to establish whether budiodarone reduces long duration AFib (duration >1 hr) in a clinical study setting using a pacemaker to evaluate the heart rhythm. The results of this analysis are provided in Table 4 below:

TABLE 4

| Episodes of AFib >1 hrs | Number | Duration | |
|---|---|---|---|
| Off Drug (baseline) | 66 | 598 | (100%) |
| 200 mg bid | 22 | 60 | (10%) |
| 400 mg bid | 12 | 56 | (9.3%) |
| 600 mg bid | 6 | 26 | (4.3%) |
| 800 mg bid | 6 | 15 | (2.5%) |

This example demonstrates a substantial reduction in the number of episodes of long duration AFib (>1 hrs) over a 2 week period. As above, “Number” indicates the number of long duration AFib episodes occurring during the 2 week period, and “Duration” indicates the number of hours during the 2 week period detected of long duration AFib episodes.

### Example 6—Reduction in Episodes of AFib Greater than 6 Minutes During a Twelve-Week Period

This example shows the robust nature of budiodarone in reducing both AFib burden and episodes of AFib greater than 6 minutes (0.1 hours) in 6 patients with either paroxysmal or persistent AFib and who previously evidenced episodes of long duration AFib (greater than 1 hr). In this example, each patient's heart rhythm was continuously monitored over a twelve-week period with a pacemaker. The patients were treated with different doses of budiodarone, with each dosage level being administered over a two-week period. In addition, during this trial, the patients were under continuous monitoring and clinician supervision.

The results are summarized in Table 5 and establish that budiodarone not only reduces long duration AFib lasting more than 1 hour in a clinical study setting using a pacemaker to evaluate the heart rhythms but also significantly lowers episodes of greater than 6 minutes (0.1 hours).

TABLE 5

| | | |
|---|---|---|
| Episodes of AFib >0.1 hrs | Number | Duration in hours |

| | | | | |
|---|---|---|---|---|
| Off Drug (baseline) | 99 | (100%) | 616 | (100%) |
| 200 mg bid | 52 | (52%) | 71 | (11%) |
| 400 mg bid | 28 | (28%) | 61 | (10%) |
| 600 mg bid | 16 | (16%) | 30 | (4.8%) |
| 800 mg bid | 15 | (15%) | 16 | (2.5%) |

The “Number” indicates the number of AFib episodes greater than 6 minutes (0.1 hour) for all tested patients over the trial period (4 weeks for baseline, 2 weeks for each experimental dosage). The “Duration” in hours indicates the cumulative duration in hours over the trial period that the tested patients were measured in episodes of AFib greater than 6 minutes. The above results demonstrate that budiodarone reduces the number of episodes of long duration AFib and reduces short episodes of AFib, thereby providing further protection against stroke and heart failure and significantly reducing the AFib burden.

For patients with paroxysmal and persistent AFib who also have high CHA₂DS₂-VASc scores (3 or higher), AFib episodes of greater than 6 minutes can lead to an increased risk of stroke and/or congestive heart failure. The CHA₂DS₂ score stands for (Congestive heart failure, Hypertension, Age (65=1 point, >75=2 points), Diabetes, and previous Stroke/transient ischemic attack (2 points). VAS stands for vascular disease (peripheral arterial disease, previous myocardial infarction, aortic atheroma), and sex category (female gender) is also included in this scoring system. The data in Table 5 evidence that, for all doses of budiodarone tested, the number of episodes of AFib longer than 6 minutes was reduced significantly, and at high concentrations of 600 mg bid or 800 mg bid, these episodes were reduced by more than 80%.

Table 6 provides a summary of the fraction of time and the number of hours per week that the patients were in episodes of AFib greater than 6 minutes in duration, on average. The percent time in AFib was reduced by 90% for doses of budiodarone at 600 mg bid and 800 mg bid.

TABLE 6

| | Fraction of | | Duration in hrs/week |
|---|---|---|---|
| Episodes of AFib >0.1 hrs | hours (percent) | | (average per patient) |
| Off Drug (baseline) | 616/4032 | (15%) | 25.2 |
| 200 mg bid | 71/2016 | (3.5%) | 5.9 |
| 400 mg bid | 61/2016 | (3.0%) | 5 |
| 600 mg bid | 30/2016 | (1.5%) | 2.5 |
| 800 mg bid | 16/2016 | (0.8%) | 1.3 |

The percent time in AFib for a patient is a critical parameter that directly relates to risk factors for stroke and/or congestive heart failure, especially for patients with high CHA₂DS₂-VASc scores. In Table 6, there was a 76.7% drop in percent time in AFib for patients using 200 mg budiodarone twice a day, which further drops to 90% for patients administered 600 mg budiodarone twice a day, and even further drops to 94.8% for patients administered 800 mg budiodarone twice a day.

### Example 7—Measurement of Longest Episodes of AFib During the Twelve-Week Period

This example is a clinical trial evaluation of 6 patients treated with different doses of budiodarone each dose tested lasting for a two-week period to determine the longest duration episode of AFib experienced at each dose. In this example, each patient's heart rhythm was continuously monitored over a twelve-week period with a pacemaker. The different doses and results of this evaluation are provided in Table 7 below:

TABLE 7

| 2 Weeks | Subject 1 | Subject 2 | Subject 3 | Subject 4 | Subject 5 | | Subject 6 | Total | |
|---|---|---|---|---|---|---|---|---|---|
| Baseline | 13 hrs | 16 hrs | 50 hrs | 5 hrs | 8 | hrs | 35 hrs | 127 | hrs |
| 200 mg bid | 5 hrs | 6 hrs | 7 hrs | 0 hrs | 3 | hrs | 6 hrs | 21 | hrs |
| 400 mg bid | 8 hrs | 13 hrs | — | 2 hrs | 2 | hrs | 10 hrs | 33 | hrs |
| 600 mg bid | 7 hrs | 0 hrs | — | 2 hrs | 0.8 | hrs | 0 hrs | 7.8 | hrs |
| 800 mg bid | 3 hrs | 5 hrs | — | 1 hr | 1.6 | hrs | 3 hrs | 13.6 | hrs |
| Washout | 105 hrs | 12 hrs | 8 hrs | 16 hrs | 2 | hrs | 18 hrs | 161 | hrs |

The above results evidence the variability of long duration AFib (when equal to or greater than 5 hours) in a patient treated with different doses of budiodarone. In Subjects 4 and 5, treatment with 200 mg budiodarone twice a day was sufficient to eliminate all episodes of long duration AFib (>5 hrs) in these patients. However, Subject 1 required 800 mg bid budiodarone to eliminate all episodes of long duration AFib (>5 hrs) whereas Subject 6 required 600 mg budiodarone bid to eliminate all episodes of budiodarone. This data establishes how long-term monitoring of a patient coupled with a dose adjustment of budiodarone is required to properly treat patients with paroxysmal or persistent AFib evidencing episodes of long duration AFib.

The results of Examples 2 to 7 establish that the use of an appropriate dose of budiodarone coupled with monitoring heart rhythms of treated patients allows for a reduction of all episodes of long duration AFib of greater than 5 hours coupled with a reduction in the overall AFib burden. These results demonstrate that a patient's progression from paroxysmal AFib to persistent AFib or from persistent AFib to permanent AFib can be reduced or prevented.

The administration of budiodarone according to the present method may be conducted using a pharmaceutical composition comprising from about 1% to 99% of budiodarone and the remainder being a pharmaceutically acceptable excipient, such as corn starch, cellulose, stearic acid, water, or other components. The pharmaceutical composition can be formulated into any form which, by way of example, only, may include one or more of a tablet, a capsule, a powder, and/or another formulation for oral administration; a parenteral administration, such as a solution suitable for one or more of intravenous administration, intramuscular administration, etc.; a suppository and/or enema; cutaneous and/or transdermal preparations, etc.

### Example 8—Elimination of Episodes of AFib Over 5.5 or 5 Hours with Longer Term Budiodarone Treatment

This example is a clinical trial evaluation of 72 patients treated with a placebo (15 patients) or 200 mg bid budiodarone (16 patients) or 400 mg bid budiodarone (16 patients) or 600 mg bid budiodarone (13 patients) for a 3-month period (12 weeks, with month 1 referring to weeks 1˜4 on the dose; month 2 referring to weeks 5-8 on the dose; and month 3 referring to weeks 9-12 on the dose) book-ended with a baseline and washout period (4 week periods before and after the 12-week experimental period and during which no budiodarone administered). In this example, the patients' heart rhythms were continuously monitored over the 5-month period of the study with a pacemaker. Table 8 shows the median duration (hours) of AFib episodes for each condition (placebo, 200 mg bid, 400 mg bid, 600 mg bid). These results demonstrate a statistically significant dose response and substantial decrease of AFib episode duration observed for all administered doses in the 2-3 months of treatment, and for the 400 mg bid and 600 mg bid even during the 1^{st} month.

TABLE 8

| Condition | Baseline | Month 1 | Month 2 | Month 3 | Washout |
|---|---|---|---|---|---|
| Placebo | 3.1 | 1.0 | 2.3 | 2.3 | 2.2 |
| 200 mg bid | 2.1 | 3.7 | 1.4 | 1.4 | 2.4 |
| 400 mg bid | 1.8 | 0.5 | 0.4 | 0.3 | 1.2 |
| 600 mg bid | 1.1 | 0.1 | 0.1 | 0.0 | 0.3 |
| P-value | 0.68 | 0.05 | 0.01 | 0.017 | 0.20 |

The P-value was calculated using the Dose-response Jonckheere-Terpstra test.

Table 9 shows more detailed results for the 600 mg bid dose relative to the placebo. Table 9 provides the number (N) of AFib episodes, mean (with standard deviation (SD)) and median duration (in hours) of AFib episodes, and maximum duration AFib episodes for the placebo control and 600 mg bid dose groups and for each month of the experiment.

TABLE 9

| | Duration of AFib episodes (hrs) | | | | |
|---|---|---|---|---|---|
| Condition | Baseline | Month 1 | Month 2 | Month 3 | Washout |
| Placebo | | | | | |
| N | 13 | 13 | 11 | 11 | 11 |
| Mean (SD) | 8.5 | 17.2 | 63.4 | 15.2 | 13.1 |
| | (19.9) | (56.8) | (202.8) | (39.7) | (33.0) |
| Median | 3.1 | 1.0 | 2.3 | 2.3 | 2.2 |
| Max | 74.2 | 206.3 | 674.9 | 133.9 | 112.0 |
| 600 mg bid | | | | | |
| N | 10 | 10 | 9 | 8 | 8 |
| Mean (SD) | 23.4 | 1.4 | 0.4 | 0.7 | 1.0 |
| | (69.6) | (2.5) | (0.7) | (1.1) | (1.6) |
| Median | 1.1 | 0.1 | 0.1 | 0.0 | 0.3 |
| Max | 221.4 | 7.1 | 2.2 | 3.1 | 4.9 |

During the month following the initiation of budiodarone administration, the average episode duration dropped to below 5 or 5.5 hours (1.4+/−0.7 (standard error) hrs). By the 2-3 months after initiation of treatment (months 2 and months 3), all episodes of AFib over 5 or 5.5 hours were eliminated, and the average episode duration dropped to below one hour (0.4+/−0.2 (standard error) hrs and 0.7+/−0.3 (standard error) hrs, respectively). Once established on budiodarone 600 mg bid in months 2 and 3 there was no patient receiving budiodarone having a high AF burden. The average AFib burden in month 2 in the treated patients was around 0.5% and the average AFib burden in month 3 for the treated patients was around 0.8%, relative to an average AFib burden at baseline of around 35%. This data demonstrates that the methods disclosed herein may be used to substantially reduce or even eliminate episodes of AFib with durations greater than 5 hours or greater than 5.5 hours. The methods may also substantially reduce or eliminate periods of substantial AFib burden, e.g., reduce or eliminate AFib burden greater than 10%, greater than 5%, or greater than 2.5% over a given 24 hour period.

### Example 9—Symptom Relief in Symptomatic Patients

This example illustrates that a AFib symptomatic patient may be evaluated for a reduction in symptoms related to AFib and/or aggregate symptoms. The patients in the experiment of Example 8 were assessed for AFib-related symptom relief via validated patient-reported outcomes. Patients were surveyed after month 3 of treatment with a placebo or at each budiodarone dose. Table 10 includes results from these patient-reported outcomes:

TABLE 10

| | | 200 mg | 400 mg | 600 mg |
|---|---|---|---|---|
| | Placebo | bid | bid | bid |
| Test Medication Control of AF | | | | |
| Symptoms | | | | |
| Made them worse | 11.8% | 0.0% | 6.3% | 0.0% |
| No change | 52.9% | 47.4% | 12.5% | 40.0% |
| Slight improvement | 17.6% | 5.3% | 31.3% | 13.3% |
| Moderate improvement | 5.9% | 21.1% | 25.0% | 0.0% |
| A lot of improvement | 11.8% | 26.3% | 25.0% | 33.3% |
| Complete relief | 0.0% | 0.0% | 0.0% | 13.3% |
| P-value (compared to placebo) | — | 0.09 | 0.03 | 0.03 |
| P-value (dose response) | 0.015 | | | |
| Satisfaction with Test Medication | | | | |
| Not at all | 41.2% | 11.1% | 6.3% | 13.3% |
| A little bit | 5.9% | 5.6% | 12.5% | 6.7% |
| Somewhat | 29.4% | 27.8% | 25.0% | 20.0% |
| A lot | 11.8% | 33.3% | 43.8% | 26.7% |
| Completely | 11.8% | 22.2% | 12.5% | 33.3% |
| P-value (compared to placebo) | — | 0.03 | 0.04 | 0.04 |

| | |
|---|---|
| P-value (dose response) | 0.03 |

The above results demonstrate that 64.7% of patients on placebo stated that the placebo either made their symptoms worse or no better as compared to an average across all three doses of budiodarone of 33%. Likewise, the number of patients stating that there was moderate, a lot, or complete relief of symptoms was 17.7% for patients on placebo, and 48% for patients averaged across all three doses of budiodarone.

The results of Table 10 show a dose-dependent improvement of self-reported AFib symptoms in response to budiodarone treatment relative to placebo. (P-value compared to placebo was calculated using the Cochran-Mantel-Haenszel mean score test; P-value dose response was calculated using the Jonckheere Terpstra test).

Patients were surveyed for specific AFib-related symptoms, including experiences of heart palpitations and shortness of breath with physical activity, for example. Patients rated symptoms on a scale from 1-6 (1=no symptoms; 2=very little; 3=a little; 4=fair amount of symptoms; 5=a lot of symptoms; 6=a great deal symptoms) and patients with >2 self-reported symptom score at baseline were included in tracking symptoms. Table 11 shows the dose-dependent improvement in these symptoms over placebo:

TABLE 11

| | | Baseline | Month 3 | Washout |
|---|---|---|---|---|
| Palpitations | Placebo | 3.5 | 3.0 | 2.4 |
| | 200 mg | 3.6 | 2.6 | 2.5 |
| | 400 mg | 4.2 | 2.7 | 3.4 |
| | 600 mg | 4.3 | 1.7 | 3.5 |
| Shortness of | Placebo | 4.0 | 3.9 | 3.7 |
| breath with | 200 mg | 3.4 | 3.3 | 3.5 |
| physical activity | 400 mg | 4.1 | 3.3 | 4.0 |
| | 600 mg | 3.3 | 2.5 | 2.4 |

### Example 10—Programming the Wearable

A wearable may be programmable (e.g., by the clinician, the patient, and/or remotely, such as by the central analysis center). The wearable may be programmed to capture the patient's heart rhythm data. The data so generated can be interrogated and/or analyzed by the CPU on the wearable and/or can be transmitted to another device for interrogation and/or analysis. One example of programming of the wearable may enable the use of the wearable for monitoring the patient's heart rhythm and relaying the data directly and/or indirectly to, e.g., a clinician as follows.

A. A USER INTERFACE: Step functions for programming AFib monitoring system involving the wearable and relying on information through a network to the attending clinician.

B. The data collected is in a readable display for the clinician and other authorized individuals to access.

C. The readable display should be made available to the authorized clinician at all times, and only the authorized clinician should be authorized to save data and/or reset the monitoring system after going through one or more of Steps 1-15 below.

D. Drug exposure, dose, and/or any other medicaments taken by the patient can be retrieved as needed by the clinician from a patient's medical records, e.g., by integrating the monitoring system with the patient's medical records (e.g., electronic medical records).

E. Steps 1 to 14 are the only manual entry required, but step 14 is optimal and can be overridden by the clinician simply pushing Manual Reset Step 15.

F. All data may be saved after review by the attending clinician, who may also be authorized to program integration of prior observation periods (Step 17), whether on or off a pharmaceutical to treat AFib including budiodarone.

G. The system is user friendly, the attending clinician only needs to do 4 things each time: 1) enter patient or code to access data, 2) review data display, 3) enter dose of budiodarone in Step 14 if they wish, then 4) Push Manual Reset and Store Data.

H. The attending clinician has access to the stored data and integrates observation periods to compare AFib characteristics on different doses of budiodarone or off-drug (Step 17).

Each of the steps is set forth in Table 12 below:

TABLE 12

| | | Units | |
|---|---|---|---|
| Step | Function or Desired Output | Displayed | Function Recorded |
| 1 | Patient Name or Code | Characters | Uncharged |
| 2 | Patient Age | Years | Unchanged |
| 3 | Observation Period | Numbers | 1-1000 |
| 3a | Observation Interval | Weeks | Minimum reset at |
| | | | least 1 week and |
| | | | at least 2 weeks |
| 4 | % Time in AFib | % | 0-100% |
| 5 | Longest Episode | Hours | Continuous variable |
| 6 | Number of episodes >24 hrs | Number | Continuous variable |
| 7 | Duration of episodes >24 hrs | Hours | Continuous variable |
| 8 | Number of episodes >5 hrs | Number | Continuous variable |
| 9 | Duration of episodes >5 hrs | Hours | Continuous variable |
| 10 | Number of episodes >1 hr | Number | Continuous variable |
| 11 | Duration of episodes >1 hr | Hours | Continuous variable |
| 12 | Number of episodes >0.1 hr | Number | Continuous variable |
| 13 | Duration of episodes >0.1 hr | Hours | Continuous variable |
| 14 | Dose of Pharmaceutical | mg/day | Optional Entry by |
| | (e.g., Budiodarone) | | Clinician |
| 15 | Push Manual Reset and Save | — | Once |
| 16 | Entry Accessed in Saved Data | All Above | Unlimited |
| 17 | Integration of Saved Data | Periods #'s | Unlimited |

Each of Steps 4-13 may be included or excluded in the programming of the wearable (e.g., at the discretion of the clinician), provided that at least one of these steps is included. In one embodiment, Steps 4-7 may be performed, and the corresponding data collected, and one or more of Steps 8-13 may be excluded. In another embodiment, Steps 4, 5, 8, and 9 may be included, and Steps 5, 6, and 10-13 may be excluded. In another embodiment, Steps 4, 5, 10, and 11 may be included, and steps 6, 7, 8, 9, 12, and 13 may be excluded. In yet another embodiment, Steps 4, 5, 12, and 13 may be included and Steps 6-11 may be excluded.

In still another embodiment, the attending clinician may program the wearable according to one or more of Steps 4-13.

List of Abbreviations:

| Abbreviation | Phrase |
|---|---|
| AAD | Anti-arrhythmic drug |
| AF or AFib | Atrial fibrillation |
| AFB | Atrial Fibrillation Burden |
| AFEQT | Atrial Fibrillation Effect on Quality of Life |
| ATP | Anti-tachycardia pacing |
| ATI-2042 | budiodarone |
| BID | Twice daily |
| CI | Confidence Interval |
| CV | Cardiovascular |
| DB | Double-blind |
| DLCO | Diffusion capacity for carbon monoxide |
| FDA | Food and Drug Administration |
| GFR | Glomerular filtration rate |
| hCE1 | Human carboxylesterase 1 |
| hERG | human Ether-à-go-go-Related channel |
| HR | Hazard Ratio |
| ICD | Implantable cardioverter defibrillator |
| ITT | Intention to treat |
| IUD | Intrauterine contraceptive device |
| LEAF | Long episodes of atrial fibrillation |
| MAFSI | Mayo Atrial Fibrillation-Specific Symptom Inventory |
| MCOT | Mobile Cardiac Outpatient Telemetry |
| mITT | Modified Intention to treat |
| NSR | Normal sinus rhythm |
| NYHA | New York Heart Association |
| QoL | Quality of Life |
| RCT | Randomized Clinical Trial |
| SCD | Sudden Cardia Death |
| VF | Ventricular fibrillation |
| VT | Ventricular tachycardia |

### EMBODIMENTS

Provided below are certain embodiments.

Embodiment I-1. A method of treating a patient diagnosed with either paroxysmal or persistent atrial fibrillation (AFib) and qualified for budiodarone therapy, said method comprising:
a) administering a first dose of budiodarone to said qualified patient while employing a wearable to monitor heart rhythm data of said qualified patient, wherein said first dose is an approved therapeutic dose that is less than a maximum approved dose of budiodarone;
b) monitoring said qualified patient for efficacy of the administered first dose of budiodarone to assess whether said first dose is efficacious for said patient during a comparison period of 1 or more days, provided that assessing the efficacy of budiodarone is delayed for at least 7 or 14 days after start of budiodarone therapy at said first dose;
c) if the first dose is assessed as not efficacious for said patient by an end of the comparison period, dose adjusting the amount of budiodarone one or more times as necessary to achieve an efficacious result using at least the same delay and comparison period as in b), provided that the adjusted dose does not exceed the maximum approved dose; or, if an efficacious result is not achieved when evaluated at the maximum approved dose, removing said patient from budiodarone therapy; and
d) if an administered dose of the first dose or an adjusted dose is efficacious for the patient, continuing to monitor the patient's heart rhythm data to ensure that the administered dose of budiodarone remains efficacious, provided that, if said administered dose is no longer efficacious, step c) is repeated with said administered dose as the first dose.

Embodiment I-2. The method of embodiment I-1, wherein said patient has been qualified for budiodarone therapy based on at least one AFib episode of at least about 5 hours or at least about 5.5 hours over at least 7 or 14 days during which said patient is not administered budiodarone therapy.

Embodiment I-3. The method of embodiment I-1, wherein said patient has been qualified for budiodarone therapy based on having a baseline level of AFib burden of at least 5 hours over at least 7 or 14 days during which said patient is not administered budiodarone therapy

Embodiment I-4. The method of any one of embodiments I-1 to I-3 wherein monitoring in step b) is conducted in a continuous manner.

Embodiment I-5. The method of any one of embodiments I-1 to I-4, wherein the efficacy of said budiodarone therapy at an administered dose is evaluated by comparing baseline levels of AFib against corresponding levels of AFib in the comparison period.

Embodiment I-6. The method of any one of embodiments I-1 to I-5, wherein the comparison period is one or more of about a day, about a week, about 1 month, about 2 months, about 3 months, about 4 months, about 5 months, or up to about 6 months.

Embodiment I-7. The method of any one of embodiments I-1 to I-5, wherein a first comparison period is followed by a second comparison period of a similar length of time.

Embodiment I-8. The method of any one of any one of embodiments I-1 to I-7, wherein episodes of AFib over about 5 hours are monitored.

Embodiment I-9. The method of any one of any one of embodiments I-1 to I-8, wherein episodes of AFib over about 5.5 hours are monitored.

Embodiment I-10. The method of any one of embodiments I-1 to I-9, wherein one or more symptoms associated with AFib are evaluated using a patient report of one or more of a number or a severity of the one or more symptoms.

Embodiment I-11. The method of any one of embodiments I-1 to I-10, wherein the budiodarone comprises a budiodarone tartrate.

Embodiment I-12. The method of any one of embodiments I-1 to I-11, further comprising, if the patient is removed from budiodarone therapy in step c), referring the patient for electrophysiological intervention

Embodiment II-1. A method of treating a patient diagnosed with either paroxysmal or persistent atrial fibrillation (AFib) and qualified for budiodarone therapy, said method comprising:
for said patient who has been qualified for budiodarone therapy, wherein said qualification requires that said patient experiences at least one episode of long duration AFib of at least 1 hour over a qualification period or has an AFib burden of at least 2.5% during the entire qualification period, wherein said qualification period is at least 2 weeks during which the patient is not treated with budiodarone:
   a) administering a first dose of budiodarone to a qualified patient while employing a wearable to monitor heart rhythm data of said qualified patient, wherein said first dose is an approved therapeutic dose that is less than a maximum approved dose for budiodarone;
   b) monitoring said patient for efficacy of the administered first dose of budiodarone to assess whether said first dose is efficacious for said patient during a comparison period of 1 or more days provided that assessing the efficacy of budiodarone is delayed for at least 7 or 14 days after start of budiodarone therapy at said first dose;
   c) if the administered first dose is assessed as not efficacious for said patient by an end of the comparison period, dose adjusting the amount of budiodarone one or more times as necessary to achieve an efficacious result using at least the same delay and comparison period as in b), provided that the adjusted dose does not exceed the maximum approved dose; or, if an efficacious result is not achieved when evaluated at the maximum approved dose, removing said patient from budiodarone therapy; and
   d) if an administered dose of the first dose or an adjusted dose is efficacious for the patient, continuing to monitor the patient's heart rhythm data to ensure that the administered dose of budiodarone remains efficacious, provided that if said administered dose is no longer efficacious step c) is repeated with said administered dose as the first dose.

Embodiment II-2. The method of embodiment II-1, wherein monitoring in step b) is conducted in a continuous manner.

Embodiment II-3. The method of any one of embodiments II-1 to II-2, wherein the efficacy of said therapy is evaluated by comparing baseline levels of AFib against corresponding levels of AFib in the comparison period.

Embodiment II-4. The method of any one of embodiments II-1 to II-3, wherein the comparison period is one or more of about a day, about a week, about 1 month, about 2 months, about 3 months, about 4 months, about 5 months, or up to about 6 months.

Embodiment II-5. The method of any one of embodiments II-1 to II-4, wherein a first comparison period is followed by a second comparison period of similar length of time.

Embodiment II-6. The method of any one of embodiments II-1 to II-5, wherein episodes of AFib over about 5 hours are monitored.

Embodiment II-7. The method of any one of embodiments II-1 to II-6, wherein episodes of AFib over about 5.5 hours are monitored.

Embodiment II-8. The method of any one of embodiments II-1 to II-7, wherein one or more symptoms associated with AFib are evaluated using a patient report of one or more of a number or a severity of the one or more symptoms.

Embodiment II-9. The method of any one of embodiments II-1 to II-8, wherein the budiodarone comprises a budiodarone tartrate or budiodarone citrate.

Embodiment II-10. The method of any one of embodiments II-1 to II-9, further comprising, if the patient is removed from budiodarone therapy in step c), referring the patient for electrophysiological intervention.

Embodiment III-1. A method of treating a patient diagnosed with either paroxysmal or persistent atrial fibrillation (AFib) and qualified for pharmacotherapy with a pharmaceutical, said method comprising:
a) administering a first dose of said pharmaceutical to said qualified patient while employing a wearable to monitor heart rhythm data of said qualified patient, wherein said first dose is an approved therapeutic dose that is less than a maximum approved dose for said pharmaceutical;
b) monitoring said patient for efficacy of the administered first dose of said pharmaceutical to assess whether said dose is efficacious for said patient during a comparison period of 1 or more days, provided that assessing the efficacy of said pharmaceutical is delayed for at least 7 or 14 days after start of pharmacotherapy with said pharmaceutical at said first dose;
c) if the first dose is assessed as not efficacious for said patient by an end of the comparison period, dose adjusting the amount of said pharmaceutical one or more times as necessary to achieve an efficacious result using at least the same delay and comparison period as in c), provided that dose adjustment does not exceed the maximum approved dose; or, if an efficacious result is not achieved when evaluated at the maximum approved dose, removing said patient from said pharmacotherapy; and
d) if an administered dose of the first dose or an adjusted dose is efficacious for the patient, continuing to monitor the patient's heart rhythm data to ensure that the administered dose of said pharmaceutical remains efficacious, provided that if said administered dose is no longer efficacious step c) is repeated with said administered dose as the first dose.

Embodiment III-2. The method of embodiment III-1, wherein said patient has been qualified for pharmacotherapy with said pharmaceutical based on at least one AFib episode of at least about 5 hours or at least about 5.5 hours over at least 7 or 14 days during which said patient is not administered said pharmaceutical.

Embodiment III-3. The method of any one of embodiments III-1 to III-2, wherein said patient has been qualified for pharmacotherapy with said pharmaceutical based on having a baseline level of AFib burden of at least 5 hours over at least 7 or 14 days during which said patient is not administered said pharmaceutical.

Embodiment III-4. The method of any one of embodiments III-1 to III-3, wherein monitoring in part c) is conducted in a continuous manner.

Embodiment III-5. The method of any one of embodiments III-1 to III-4, wherein the efficacy of said pharmacotherapy with the pharmaceutical at an administered dose is evaluated by comparing baseline levels of AFib against corresponding levels of AFib in the comparison period.

Embodiment III-6. The method of any one of embodiments III-1 to III-5, wherein the comparison period is one or more of about a day, about a week, about 1 month, about 2 months, about 3 months, about 4 months, about 5 months, or up to about 6 months.

Embodiment III-7. The method of any one of embodiments III-1 to III-6, wherein a first comparison period is followed by a second comparison period of similar length of time.

Embodiment III-8. The method of any one of any one of embodiments III-1 to 111-7, wherein episodes of AFib over about 5 hours are monitored.

Embodiment III-9. The method of any one of any one of embodiments III-1 to 111-8, wherein episodes of AFib over about 5.5 hours are monitored.

Embodiment III-10. The method of any one of embodiments III-1 to III-9, wherein one or more symptoms associated with AFib are evaluated using a patient report of one or more of a number or a severity of the one or more symptoms.

Embodiment III-11. The method of any one of embodiments III-1 to III-10, further comprising, if the patient is removed from the pharmacotherapy in step c), referring the patient for electrophysiological intervention.

Embodiment IV-1. A method of treating a patient diagnosed with either paroxysmal or persistent atrial fibrillation (AFib) and qualified for pharmacotherapy with a pharmaceutical, said method comprising:
for said patient who has been qualified for pharmacotherapy with said pharmaceutical, wherein said qualification requires that said patient experiences at least one episode of long duration AFib of at least 1 hour during a qualification period or has an AFib burden of at least 2.5% during the entire qualification period, wherein said qualification period is at least 2 weeks during which the patient is not treated with said pharmaceutical;
   a) administering a first dose of said pharmaceutical to the qualified patient while employing a wearable to monitor heart rhythm data of said qualified patient, wherein said first dose is an approved therapeutic dose which is less than a maximum approved dose for said pharmaceutical;
   b) monitoring said patient for efficacy of the administered first dose of said pharmaceutical to assess whether said first dose is efficacious for said patient during a comparison period of 1 or more days provided that assessing the efficacy of said pharmaceutical is delayed for at least 7 or 14 days after start of said pharmacotherapy at said first dose;
   c) if the administered first dose is assessed as not efficacious for said patient by an end of the comparison period, dose adjusting the amount of said pharmaceutical one or more times as necessary to achieve an efficacious result using at least the same delay and comparison period as in b), provided that the adjusted dose does not exceed the maximum approved dose; or if an efficacious result is not achieved when evaluated at the maximum approved dose, removing said patient from said pharmacotherapy; and
   d) if an administered dose of the first dose or an adjusted dose is efficacious for the patient, continuing to monitor the patient's heart rhythm data to ensure that the administered dose of said pharmaceutical remains efficacious, provided that if said administered dose is no longer efficacious, step c) is repeated with said administered dose as the first dose.

Embodiment IV-2. The method of embodiment IV-1, wherein monitoring in step b) is conducted in a continuous manner.

Embodiment IV-3. The method of any one of embodiments IV-1 to IV-2, wherein the efficacy of said pharmacotherapy with the pharmaceutical at an administered dose is evaluated by comparing baseline levels of AFib against corresponding levels of AFib in the comparison period.

Embodiment IV-4. The method of any one of embodiments IV-1 to IV-3, wherein the comparison period is one or more of about a day, about a week, about 1 month, about 2 months, about 3 months, about 4 months, about 5 months, or up to about 6 months.

Embodiment IV-5. The method of any one of embodiments IV-1 to IV-4, wherein a first comparison period is followed by a second comparison period of a similar length of time.

Embodiment IV-6. The method of any one of embodiments IV-1 to IV-5, wherein episodes of AFib over about 5 hours are monitored.

Embodiment IV-7. The method of any one of embodiments IV-1 to IV-6, wherein episodes of AFib over about 5.5 hours are monitored.

Embodiment IV-8. The method of any one of embodiments IV-1 to IV-7, wherein one or more symptoms associated with AFib are evaluated using a patient report of one or more of a number or a severity of the one or more symptoms.

Embodiment IV-9. The method of any one of embodiments IV-1 to IV-8, further comprising, if the patient is removed from pharmacotherapy in step c), referring the patient for electrophysiological intervention.

What is claimed is:

1. A method of treating a patient diagnosed with either paroxysmal or persistent atrial fibrillation (AFib) and qualified for budiodarone therapy, said method comprising:
a) administering a first dose of budiodarone to said qualified patient while employing a wearable to monitor heart rhythm data of said qualified patient, wherein said first dose is an approved therapeutic dose that is less than a maximum approved dose of budiodarone;
b) monitoring said qualified patient for efficacy of the administered first dose of budiodarone to assess whether said first dose is efficacious for said patient during a comparison period of 1 or more days, provided that assessing the efficacy of budiodarone is delayed for at least 7 or 14 days after start of budiodarone therapy at said first dose;
c) if the first dose is assessed as not efficacious for said patient by an end of the comparison period, dose adjusting the amount of budiodarone one or more times as necessary to achieve an efficacious result using at least the same delay and comparison period as in b), provided that the adjusted dose does not exceed the maximum approved dose; or, if an efficacious result is not achieved when evaluated at the maximum approved dose, removing said patient from budiodarone therapy; and
d) if an administered dose of the first dose or an adjusted dose is efficacious for the patient, continuing to monitor the patient's heart rhythm data to ensure that the administered dose of budiodarone remains efficacious, provided that, if said administered dose is no longer efficacious, step c) is repeated with said administered dose as the first dose.

2. The method of claim 1, wherein said patient has been qualified for budiodarone therapy based on at least one AFib episode of at least about 5 hours or at least about 5.5 hours over at least 7 or 14 days during which said patient is not administered budiodarone therapy.

3. The method of claim 1, wherein said patient has been qualified for budiodarone therapy based on having a baseline level of AFib burden of at least 5 hours over at least 7 or 14 days during which said patient is not administered budiodarone therapy.

4. The method of any one of claim 1, 2 or 3, wherein monitoring in step b) is conducted in a continuous manner.

5. The method of claim 4, wherein the efficacy of said budiodarone therapy at an administered dose is evaluated by comparing baseline levels of AFib against corresponding levels of AFib in the comparison period.

6. The method of claim 5, wherein the comparison period is one or more of about a day, about a week, about 1 month, about 2 months, about 3 months, about 4 months, about 5 months, or up to about 6 months.

7. The method of claim 6, wherein a first comparison period is followed by a second comparison period of a similar length of time.

8. The method of any one of claim 1, 2 or 3, wherein episodes of AFib over about 5 hours are monitored.

9. The method of any one of claim 1, 2 or 3, wherein episodes of AFib over about 5.5 hours are monitored.

10. The method of any one of claim 1, 2 or 3, wherein one or more symptoms associated with AFib are evaluated using a patient report of one or more of a number or a severity of the one or more symptoms.

11. The method of any one of claim 1, 2 or 3, wherein the budiodarone comprises a budiodarone tartrate.

12. The method of any one of claim 1, 2 or 3, further comprising, if the patient is removed from budiodarone therapy in step c), referring the patient for electrophysiological intervention.

13. A method of treating a patient diagnosed with either paroxysmal or persistent atrial fibrillation (AFib) and qualified for budiodarone therapy, said method comprising:
for said patient who has been qualified for budiodarone therapy, wherein said qualification requires that said patient experiences at least one episode of long duration AFib of at least 1 hour over a qualification period or has an AFib burden of at least 2.5% during the entire qualification period, wherein said qualification period is at least 2 weeks during which the patient is not treated with budiodarone:
a) administering a first dose of budiodarone to said qualified patient while employing a wearable to monitor heart rhythm data of said qualified patient, wherein said first dose is an approved therapeutic dose that is less than a maximum approved dose for budiodarone;
b) monitoring said patient for efficacy of the administered first dose of budiodarone to assess whether said first dose is efficacious for said patient during a comparison period of 1 or more days, provided that assessing the efficacy of budiodarone is delayed for at least 7 or 14 days after start of budiodarone therapy at said first dose;
c) if the administered first dose is assessed as not efficacious for said patient by an end of the comparison period, dose adjusting the amount of budiodarone one or more times as necessary to achieve an efficacious result using at least the same delay and comparison period as in b), provided that the adjusted dose does not exceed the maximum approved dose; or, if an efficacious result is not achieved when evaluated at the maximum approved dose, removing said patient from budiodarone therapy; and
d) if an administered dose of the first dose or an adjusted dose is efficacious for the patient, continuing to monitor the patient's heart rhythm data to ensure that the administered dose of budiodarone remains efficacious, provided that, if said administered dose is no longer efficacious, step c) is repeated with said administered dose as the first dose.

14. The method of claim 13, wherein monitoring in step b) is conducted in a continuous manner.

15. The method of claim 13, wherein the efficacy of said budiodarone therapy at an administered dose is evaluated by comparing baseline levels of AFib against corresponding levels of AFib in the comparison period.

16. The method of claim 13, wherein the comparison period is one or more of about a day, about a week, about 1 month, about 2 months, about 3 months, about 4 months, about 5 months, or up to about 6 months.

17. The method of claim 16, wherein a first comparison period is followed by a second comparison period of similar length of time.

18. The method of claim 13, wherein episodes of AFib over about 5 hours are monitored.

19. The method of claim 13, wherein episodes of AFib over about 5.5 hours are monitored.

20. The method of claim 13, wherein one or more symptoms associated with AFib are evaluated by the patient using a patient report of one or more of a number or a severity of the one or more symptoms.

21. The method of claim 13, wherein the budiodarone comprises a budiodarone tartrate.

22. The method of claim 13, further comprising, if the patient is removed from budiodarone therapy in step c), referring the patient for electrophysiological intervention.

23. A method of treating a patient diagnosed with either paroxysmal or persistent atrial fibrillation (AFib) and qualified for pharmacotherapy with a pharmaceutical, said method comprising:
a) administering a first dose of said pharmaceutical to said qualified patient while employing a wearable to monitor heart rhythm data of said qualified patient, wherein said first dose is an approved therapeutic dose that is less than a maximum approved dose for said pharmaceutical;
b) monitoring said patient for efficacy of the administered first dose of said pharmaceutical to assess whether said dose is efficacious for said patient during a comparison period of 1 or more days, provided that assessing the efficacy of said pharmaceutical is delayed for at least 7 or 14 days after start of pharmacotherapy with said pharmaceutical at said first dose;
c) if the first dose is assessed as not efficacious for said patient by an end of the comparison period, dose adjusting the amount of said pharmaceutical one or more times as necessary to achieve an efficacious result using at least the same delay and comparison period as in c), provided that dose adjustment does not exceed the maximum approved dose; or, if an efficacious result is not achieved when evaluated at the maximum approved dose, removing said patient from said pharmacotherapy; and
d) if an administered dose of the first dose or an adjusted dose is efficacious for the patient, continuing to monitor the patient's heart rhythm data to ensure that the administered dose of said pharmaceutical remains efficacious, provided that, if said administered dose is no longer efficacious, step c) is repeated with said administered dose as the first dose.

24. The method of claim 23, wherein said patient has been qualified for pharmacotherapy with said pharmaceutical based on at least one AFib episode of at least about 5 hours or at least about 5.5 hours over at least 7 or 14 days during which said patient is not administered said pharmaceutical.

25. The method of claim 23, wherein said patient has been qualified for pharmacotherapy with said pharmaceutical based on having a baseline level of AFib burden of at least 5 hours over at least 7 or 14 days during which said patient is not administered said pharmaceutical.

26. The method of any one of claim 23, 24 or 25, wherein monitoring in part c) is conducted in a continuous manner.

27. The method of any one of claim 23, 24 or 25, wherein the efficacy of said pharmacotherapy with the pharmaceutical at an administered dose is evaluated by comparing baseline levels of AFib against corresponding levels of AFib in the comparison period.

28. The method of claim 27, wherein the comparison period is one or more of about a day, about a week, about 1 month, about 2 months, about 3 months, about 4 months, about 5 months, or up to about 6 months.

29. The method of claim 28, wherein a first comparison period is followed by a second comparison period of similar length of time.

30. The method of any one of claim 23, 24 or 25, wherein episodes of AFib over about 5 hours are monitored.

31. The method of any one of claim 23, 24 or 25, wherein episodes of AFib over about 5.5 hours are monitored.

32. The method of any one of claim 23, 24 or 25, wherein one or more symptoms associated with AFib are evaluated using a patient report of one or more of a number or a severity of the one or more symptoms.

33. The method of any one of claim 23, 24 or 25, further comprising, if the patient is removed from the pharmacotherapy in step c), referring the patient for electrophysiological intervention.

34. A method of treating a patient diagnosed with either paroxysmal or persistent atrial fibrillation (AFib) and qualified for pharmacotherapy with a pharmaceutical, said method comprising:
for said patient who has been qualified for pharmacotherapy with said pharmaceutical, wherein said qualification requires that said patient experiences at least one episode of long duration AFib of at least 1 hour during a qualification period or has an AFib burden of at least 2.5% during the entire qualification period, wherein said qualification period is at least 2 weeks during which the patient is not treated with said pharmaceutical;
a) administering a first dose of said pharmaceutical to the qualified patient while employing a wearable to monitor heart rhythm data of said qualified patient, wherein said first dose is an approved therapeutic dose which is less than a maximum approved dose for said pharmaceutical;
b) monitoring said patient for efficacy of the administered first dose of said pharmaceutical to assess whether said first dose is efficacious for said patient during a comparison period of 1 or more days provided that assessing the efficacy of said pharmaceutical is delayed for at least 7 or 14 days after start of said pharmacotherapy at said first dose;
c) if the administered first dose is assessed as not efficacious for said patient by an end of the comparison period, dose adjusting the amount of said pharmaceutical one or more times as necessary to achieve an efficacious result using at least the same delay and comparison period as in b), provided that the adjusted dose does not exceed the maximum approved dose; or if an efficacious result is not achieved when evaluated at the maximum approved dose, removing said patient from said pharmacotherapy; and
d) if an administered dose of the first dose or an adjusted dose is efficacious for the patient, continuing to monitor the patient's heart rhythm data to ensure that the administered dose of said pharmaceutical remains efficacious, provided that, if said administered dose is no longer efficacious, step c) is repeated with said administered dose as the first dose.

35. The method of claim 34, wherein monitoring in step b) is conducted in a continuous manner.

36. The method of claim 34, wherein the efficacy of said pharmacotherapy with the pharmaceutical at an administered dose is evaluated by comparing baseline levels of AFib against corresponding levels of AFib in the comparison period.

37. The method of claim 34, wherein the comparison period is one or more of about a day, about a week, about 1 month, about 2 months, about 3 months, about 4 months, about 5 months, or up to about 6 months.

38. The method of claim 37, wherein a first comparison period is followed by a second comparison period of a similar length of time.

39. The method of claim 34, wherein episodes of AFib over about 5 hours are monitored.

40. The method of claim 34, wherein episodes of AFib over about 5.5 hours are monitored.

41. The method of claim 34, wherein one or more symptoms associated with AFib are evaluated using a patient report of one or more of a number or a severity of the one or more symptoms.

42. The method of claim 34, further comprising, if the patient is removed from pharmacotherapy in step c), referring the patient for electrophysiological intervention.
